(19) United States
(12) Patent Application Publication
(10) Pub. No.: US 2026/0017696 A1
(43) Pub. Date: Jan. 15, 2026

(54) Determining Temporal Parameters For Evaluating Outpatient Facilities Based On Complication Rates

(71) Applicant: Healthgrades Marketplace, LLC, Denver, CO (US)

(72) Inventors: Aimee GATCHALIAN, Denver, CO (US); Brad BOWMAN, Denver, CO (US); Zach EHASZ, Denver, CO (US); Thomas JACKSON, Denver, CO (US); Soo RHEE, Denver, CO (US); Sean WALLACE, Denver, CO (US)

(73) Assignee: Healthgrades Marketplace, LLC, Denver, CO (US)

(21) Appl. No.: 18/657,641

(22) Filed: May 7, 2024

Related U.S. Application Data
(60) Provisional application No. 63/464,934, filed on May 8, 2023.

(51) Int. Cl. G06Q 30/0282; G16H 10/60; G16H 40/20

(52) U.S. Cl. CPC G06Q 30/0282; G16H 10/60; G16H 40/20

(57) ABSTRACT

A method for rating a medical facility based on complication rates includes determining, for each procedure of a group of procedures, a temporal filter for complications associated with the procedure based on information obtained from a group of data sources, the group data sources including one or more data sources with encrypted patient data. The method also includes identifying a group of patients having performed a same procedure at the medical facility. The method further includes filtering, from the group of patients, complications associated with the group of patients based on the temporal filter to generate a list of filtered complications. The method also includes generating a rating for the medical facility based on a quantity of complications in the list of filtered complications.

### CROSS-REFERENCE TO RELATED APPLICATION

The present application claims the benefit of U.S. Provisional Patent Application No. 63/464,934, filed on May 8, 2023, and titled “EVALUATING OUTPATIENT FACILITIES BASED ON COMPLICATION RATES DETERMINED USING TEMPORAL PARAMETERS,” the disclosure of which is expressly incorporated by reference in its entirety.

### FIELD

The present disclosure relates generally to evaluating outpatient clinical surgical facilities, and more specifically to determining temporal parameters for evaluating outpatient clinical facilities based on complications rates associated with specific procedures or treatments.

### BACKGROUND

Individuals undergoing surgical procedures or treatments generally seek to learn as much information as possible about the facility where the procedure/treatment will be performed. This is particularly true when the health care provider who will perform the procedure or treatment(s) has privileges at multiple facilities and may perform the work in both inpatient and outpatient settings.

In a traditional hospital stay, there is extensive documentation of the patient's health upon arrival, during the procedure or treatment, in recovery throughout the hospital stay, and at discharge. Outpatient facilities, however, do not undertake the same level of documentation. An outpatient facility is a type of healthcare facility where patients receive medical care without being admitted to a hospital or staying overnight. These facilities offer a range of services, including consultations, diagnostic tests, treatments, surgeries, and therapies, among others. Outpatient facilities can vary widely in size and specialization, from small clinics and medical offices to large outpatient surgery centers and specialized treatment centers. They provide a convenient and cost-effective option for patients who do not require overnight hospitalization but still need medical attention and services.

Depending on the procedure or treatment, comorbidities, and patient health parameters upon arrival may not be fully recorded at outpatient facilities. In addition, because release from outpatient facilities is typically the same day as the procedure or treatment, health information is limited to what is recorded during the procedure or treatment and during the short stay at the facility. It is known, however, that complications from a given procedure or treatment may not arise until days, weeks, or even months after the patient is discharged from the outpatient facility. Such complications thus may not be detected and/or linked to the performance of a procedure or treatment.

### SUMMARY

In aspects of the present disclosure, a method for rating a medical facility based on complication rates includes determining, for each procedure of a group of procedures, a temporal filter for complications associated with the procedure based on information obtained from a group of data sources, the group data sources including one or more data sources with encrypted patient data. The method further includes identifying a group of patients having performed a same procedure at the medical facility. The method also includes filtering, from the group of patients, complications associated with the group of patients based on the temporal filter to generate a list of filtered complications. The method further includes generating a rating for the medical facility based on a quantity of complications in the list of filtered complications.

Other aspects of the present disclosure are directed to an apparatus. The apparatus includes means for determining, for each procedure of a group of procedures, a temporal filter for complications associated with the procedure based on information obtained from a group of data sources, the group data sources including one or more data sources with encrypted patient data. The apparatus further includes means for identifying a group of patients having performed a same procedure at the medical facility. The apparatus also includes means for filtering, from the group of patients, complications associated with the group of patients based on the temporal filter to generate a list of filtered complications. The apparatus further includes means for generating a rating for the medical facility based on a quantity of complications in the list of filtered complications.

In other aspects of the present disclosure, a non-transitory computer-readable medium with program code recorded thereon is disclosed. The program code is executed by a processor and includes program code to determine, for each procedure of a group of procedures, a temporal filter for complications associated with the procedure based on information obtained from a group of data sources, the group data sources including one or more data sources with encrypted patient data. The program code further includes program code to identify a group of patients having performed a same procedure at the medical facility. The program code also includes program code to filter, from the group of patients, complications associated with the group of patients based on the temporal filter to generate a list of filtered complications. The program code further includes program code to generate a rating for the medical facility based on a quantity of complications in the list of filtered complications.

Other aspects of the present disclosure are directed to an apparatus. The apparatus includes one or more memories coupled with the one or more processors and storing processor-executable code that, when executed by the one or more processors, is configured to cause the apparatus to determine, for each procedure of a group of procedures, a temporal filter for complications associated with the procedure based on information obtained from a group of data sources, the group data sources including one or more data sources with encrypted patient data. Execution of the processor-executable code further causes the apparatus to identify a group of patients having performed a same procedure at the medical facility. Execution of the processor-executable code also causes the apparatus to filter, from the group of patients, complications associated with the group of patients based on the temporal filter to generate a list of filtered complications. Execution of the processor-executable code still further causes the apparatus to generate a rating for the medical facility based on a quantity of complications in the list of filtered complications.

Additional features and advantages of the disclosure will be described below. It should be appreciated by those skilled in the art that this disclosure may be readily utilized as a basis for modifying or designing other structures for carrying out the same purposes of the present disclosure. It should also be realized by those skilled in the art that such equivalent constructions do not depart from the teachings of the disclosure as set forth in the appended claims. The novel features, which are believed to be characteristic of the disclosure, both as to its organization and method of operation, together with further objects and advantages, will be better understood from the following description when considered in connection with the accompanying figures. It is to be expressly understood, however, that each of the figures is provided for the purpose of illustration and description only and is not intended as a definition of the limits of the present disclosure.

### BRIEF DESCRIPTION OF THE DRAWINGS

So that features of the present disclosure can be understood in detail, a particular description may be had by reference to aspects, some of which are illustrated in the appended drawings. It is to be noted, however, that the appended drawings illustrate only certain aspects of this disclosure and are therefore not to be considered limiting of its scope, for the description may admit to other equally effective aspects. The same reference numbers in different drawings may identify the same or similar elements.

FIG. 1 is a flow diagram illustrating an example of a network environment by which complication results from the computer system are obtained, in accordance with aspects of the present disclosure.

FIG. 2A is a schematic illustrating an example of a webpage through which complication data may be obtained, in accordance with aspects of the present disclosure.

FIG. 2B is a block diagram illustrating a process for anonymizing and configuring outpatient encounter data, in accordance with aspects of the present disclosure.

FIG. 3A is a block diagram illustrating an example of the analysis performed for including a particular complication into the complication rate determination, in accordance with aspects of the present disclosure.

FIG. 3B is a block diagram illustrating the risk adjustment process used to score a facility based on its complication rates, and patient case mix, in accordance with aspects of the present disclosure.

FIG. 4 illustrates an example of a search page used to identify complication rates by procedure or treatment, in accordance with an embodiment of the present application.

FIG. 5 illustrates an example of scoring that may be used to reflect complication rate results following a search for a particular procedure or treatment at a specific outpatient facility, in accordance with an embodiment of the present application.

FIG. 6 illustrates results obtained by a search for outpatient facilities having a particular ranking awarded on the basis of complication rates, in accordance with aspects of the present disclosure.

FIG. 7 is a flow diagram illustrating an example of a process for filtering complications in order to determine a number of complications associated with an outpatient facilitate, in accordance with various aspects of the present disclosure.

### DETAILED DESCRIPTION

The detailed description set forth below, in connection with the drawings, is intended as a description of various configurations and is not intended to represent the only configurations in which the concepts described herein may be practiced. The detailed description include specific details for the purpose of providing a thorough understanding of the various concepts. It will be apparent to those skilled in the art, however, that these concepts may be practiced without these specific details. In some instances, well-known structures and components are shown in block diagram form in order to avoid obscuring such concepts.

Based on the teachings, one skilled in the art should appreciate that the scope of the present disclosure is intended to cover any aspect of the present disclosure, whether implemented independently of or combined with any other aspect of the present disclosure. For example, an apparatus may be implemented, or a method may be practiced using any number of the aspects set forth. In addition, the scope of the present disclosure is intended to cover such an apparatus or method practiced using other structure, functionality, or structure and functionality in addition to, or other than the various aspects of the present disclosure set forth. It should be understood that any aspect of the present disclosure may be embodied by one or more elements of a claim.

The word “exemplary” is used herein to mean “serving as an example, instance, or illustration.” Any aspect described herein as “exemplary” is not necessarily to be construed as preferred or advantageous over other aspects.

Although particular aspects are described herein, many variations and permutations of these aspects fall within the scope of the present disclosure. Although some benefits and advantages of the preferred aspects are mentioned, the scope of the present disclosure is not intended to be limited to particular benefits, uses, or objectives. Rather, aspects of the present disclosure are intended to be broadly applicable to different technologies, system configurations, networks, and protocols, some of which are illustrated by way of example in the figures and in the following description of the preferred aspects. The detailed description and the drawings are merely illustrative of the present disclosure rather than limiting, the scope of the present disclosure being defined by the appended claims and equivalents thereof.

Individuals undergoing surgical procedures or treatments generally seek to learn as much information as possible about the facility where the procedure/treatment will be performed. This is particularly true when the health care provider who will perform the procedure or treatment(s) has privileges at multiple facilities and may perform the work in both inpatient and outpatient settings.

While there currently are computer systems that apply quantitative and non-subjective factors to evaluate outcomes of inpatient procedures at hospitals, there is no similar system available that can effectively and objectively evaluate outpatient complications. There are several obstacles to overcome when evaluating patient outcomes of procedures or treatments at outpatient facilities. In a traditional hospital stay, there is extensive documentation of the patient's health upon arrival, during the procedure or treatment, in recovery throughout the hospital stay, and at discharge. Outpatient facilities, however, do not undertake the same level of documentation. Depending on the procedure or treatment, comorbidities, and patient health parameters upon arrival may not be fully recorded at outpatient facilities. In addition, because release from outpatient facilities is typically the same day as the procedure or treatment, health information is limited to what is recorded during the procedure or treatment and during the short stay at the facility. It is known, however, that complications from a given procedure or treatment may not arise until days, weeks, or even months after the patient is discharged from the outpatient facility. Such complications thus may not be detected and/or linked to the performance of a procedure or treatment.

Relating a given complication to an outpatient procedure or treatment is further hindered based on how medical information is collected and stored, particularly in the United States. In the U.S., patient health records from all providers for a given patient are not compiled and combined into a comprehensive health record or registry. A patient who has undergone a procedure or treatment at an outpatient facility may not return to that facility for complications. Instead, the patient may visit, for example, a general practitioner, an emergency clinical setting, their referring physician or surgeon who performed the surgery, another provider, etc. Records for each of these providers and facilities used by the patient experiencing complications are almost always monolithic and not shared. The provider or facility often may not know about prior procedures or treatments performed on a patient or comorbidities identified by other providers. Additionally, the patient may not even identify a particular complication as a possible result of the procedure or treatment received.

For these reasons, current analysis of procedures and treatments at outpatient facilities relies heavily on patient surveys, perceived reputational rankings of the facility, and other factors that are subjective and do not directly assess medical outcomes. Such analysis is unreliable and unlikely to provide a clear picture regarding the abilities of the outpatient facilities compared to the average outpatient facility performing the same procedure or treatment.

The absence of a reliable and reproducible methodology for evaluating outpatient facilities outcomes presents real world problems that affect several aspects of healthcare. It hampers the ability of health care providers and health care employees to compare and select appropriate facilities when they are deciding where to work, and/or obtain or maintain privileges. Having a system that permits useful comparative evaluations for outpatient facilities further allows the community, the facility owners, regulatory bodies, and the like to identify poorly performing facilities for audits, improvement plans, or closures. Alternatively, a computer system that allows for the identification of facilities that are excelling may benefit from the exposure, attracting better providers and employees, increased investment, or other advantages deserved by the outperforming facility.

An outpatient facility can also use a robust and reliable system of evaluating outpatient facilities to track its own historical performance and determine if there is an improvement or a decline in patient outcomes. Further, insurers may use the system to identify which outpatient facilities should be in-network, and the management of groups of affiliated facilities may use the system to identify outpatient facilities that should be acquired or divested.

There exists a need for a computer system and methodology that correctly identifies when complications are related to the procedures or treatments performed in an outpatient setting. Such procedures or treatments could be of any kind, such as surgeries, treatment for acute medical conditions (heart attack, stroke), or the management of a health condition where outpatient facilities provide the necessary care. There further exists a need for a computer system and methodology that evaluates complication rates in a quantifiable, reliable, verifiable, reproducible, transparent, and objective manner.

Various aspects of the present disclosure are directed to evaluating outpatient clinical facilities based on the rate of complications associated with specific procedures or treatments. Whether the complication is related to the procedure or treatment is determined using time bands, which are examples of temporal filters that may be determined from a corpus of data sources, such as, but not limited to patient health records, medical billings claims data, treatises, authoritative resources, experts in the field. These data sources may be gathered from various data mining techniques, including, but not limited to, data scraping and/or accessing third party data systems. In some cases, the temporal filters may be autonomously determined and dynamically updated via machine learning techniques. Various aspects of the present disclosure also facilitate comparative evaluations among multiple outpatient facilities for one or more procedures/treatments or generally as a whole. The system also uses risk adjustment to account for differences in the health of patient populations at one outpatient facility compared to another, permitting fair statistical comparisons of complication rates.

In some examples, a computer system is provided where complications at an individual outpatient facility may be evaluatively compared and/or ranked vis-à-vis complications arising out of a particular cohort of outpatient facilities. In other examples, a computer system is provided where complications for a selected group of outpatient facilities can be evaluated against a particular cohort.

The comparative facility cohort can be developed from any set of outpatient facilities so long as the cohort is sufficiently robust to provide reliable data on procedure or treatment outcomes. As non-limiting examples of cohorts that may be used for comparative purposes and establishing baseline complication rates, the computer system can identify and aggregate outcomes at outpatient facilities that are national or even worldwide. Alternatively, the comparative cohort may include facilities that are: within a particular geographic area; facilities that have a particular specialty or a specific patient population (e.g., pediatric, veterans, geriatric care, etc.); facilities that are within a particular insurance network; and/or facilities are affiliated with a particular hospital or clinical network.

To overcome the limitations of data collection at an outpatient facility, e.g., limited patient intake information and a short period for observing and recording health effects associated with the procedure, the computer system may learn patient comorbidities (risk factors) and clinical outcomes such as deaths and clinical complications based on data derived from electronic medical record (EMR) extracts, billing and financial information, including but not limited to, Medicare (MedPar and Standard Analytics File) data sets, and/or state and commercial all-payer claims. In one embodiment, data from Medicare patient health records and/or Medicare medical billing claims data is used. In some examples, such data is not readily accessible to a human and is encrypted. Therefore, such information may only be accessed via special access privileges. Medicare patient health records track individual patients across all providers participating in Medicare. The vast majority of health care providers and facilities offer Medicare services; moreover, Medicare patients are unlikely to seek non-Medicare providers given the extra cost they will face. Medicare patient health records thus can provide a comprehensive data set providing information about the patient that may not have been recorded at the outpatient facility. This longitudinal data set can be used to correlate the procedure performed on a patient with complications developing after release from the outpatient facilities days, weeks, months, or even years after the patient undergoes the procedure.

Other sources of health data may also provide data sufficient for the computer system disclosed herein. For example, some countries or localities have nationalized patient medical records that provide longitudinal and comprehensive health information across providers by patient. The use of such records would enable the claimed system to reliably develop complication rates associated with particular outpatient procedures.

In addition, the system may use patient health records and/or medical billing claims data from particular health insurers. These files may be licensed from or provided by the Centers for Medicare and Medicaid Services, individual states, a healthcare system, individual facilities, and 3rd party data aggregators. The files may be provided as individual data files via SFTP transfers, or access may be provided via application programming interfaces (APIs). All patient identifiers are removed from the files beforehand. The data for each anonymous user is grouped by patient admissions or appended together to enable longitudinal tracking of individual patient medical encounters over time. For example, HMOs require participants to see in-network providers for reimbursement and insurance benefits. The in-network visits are compiled and recorded by individuals as part of medical billing claims. Because treatment by out-of-network providers is not covered, many participants have their health needs covered by in-network providers monitored by the HMOs. This is particularly true in the case of complications, which may require expensive follow-on procedures, specialists, and care. HMO-derived health data may thus be used to train the computer system and evaluate outpatient facility metrics.

Data from PPOs may also prove sufficient for the system. PPOs often have and encourage the use of in-network providers but also sometimes provide discounts and caps when out-of-network providers are used. Longitudinal tracking thus may be possible based on medical billings claims data for patients that use in-network providers and out-of-network providers for which reimbursement is available.

Other sources of health data may also provide data sufficient for the computer system disclosed herein. For example, some countries or localities have nationalized patient medical records that provide longitudinal and comprehensive health information across providers by patient. Use of such records would enable the claimed system to reliably develop complication rates associated with particular outpatient procedures.

In addition, the system may use patient health records and/or medical billing claims data from particular health insurers. These files may be licensed from or provided by the Centers for Medicare and Medicaid Services, individual states, a healthcare system, individual facilities, and 3rd party data aggregators. The files may be provided as individual data files via SFTP transfers, or access may be provided via application programming interfaces (APIs). All patient identifiers are removed from the files beforehand. The data for each anonymous user is grouped by patient admissions or appended together to enable longitudinal tracking of individual patient medical encounters over time. For example, HMOs require participants to see in-network providers for reimbursement and insurance benefits. The in-network visits are compiled and recorded by individuals as part of medical billing claims. Because treatment by out-of-network providers is not covered, many participants have their health needs covered by in-network providers monitored by the HMOs. This is particularly true in the case of complications, which may require expensive follow-on procedures, specialists, and care. HMO-derived health data may thus be used to train the computer system and evaluate outpatient facility metrics.

Data from PPOs may also prove sufficient for the system. PPOs often have and encourage the use of in-network providers but also sometimes provide discounts and caps when out-of-network providers are used. Longitudinal tracking thus may be possible based on medical billings claims data for patients that use in-network providers and out-of-network providers for which reimbursement is available.

As discussed, various aspects of the present disclosure overcome data limitations in outpatient facilities through a multi-faceted approach that leverages various data sources and encryption protocols. In some examples, electronic medical records (EMR) may be analyzed to obtain patient information ranging from medical history to treatment outcomes. This data, coupled with billing and financial information, offers insights into patient comorbidities (e.g., risk factors) and clinical outcomes, such as death and complications. The system may also access Medicare data sets such as MedPar and Standard Analytics File, which provide comprehensive health records and billing claims for Medicare patients across different providers. State and commercial all-payer claims data further enriches the analysis, offering a broader perspective on patient care and outcomes. To ensure data security and privacy, sensitive information, such as encrypted patient data, is accessible only via special access privileges, and patient identifiers are removed from files before analysis.

In some examples, the system correlates procedures with complications over extended periods post-treatment. The system may also capitalize on nationalized patient medical records in some regions, providing comprehensive health data crucial for assessing complication rates associated with outpatient procedures accurately. Moreover, the system integrates health insurer data from sources, such as HMOs and PPOs, offering insights into patient behavior regarding in-network and out-of-network providers, reimbursement policies, and their impact on complication rates. Given the vast amount of data, such correlation procedures could not be performed by a human.

The system may use the data (e.g., data collected from various online sources) to train a machine learning model or another type of function (e.g., temporal filter function) to understand patterns, risk factors, and correlations related to different procedures and patient demographics. Based on the patterns, risk factors, and correlations related to different procedures and patient demographics, the machine learning model or temporal filter function determines a temporal filter for each procedure and patient demographic.

In some examples, clinical input is used to establish and/or update the temporal filters. In such examples, the clinical input may include one or more medical codes, where each medical code is associated with a medical procedure and a time when the medical procedure was performed. A machine learning model may be used to examine the distribution of medical codes within these proposed time bands. The machine learning function may identify any medical codes whose distribution falls outside the boundaries of the temporal filter. This step identifies potential discrepancies or outliers that may need further investigation. Following this initial analysis, clinical input is again leveraged to assess the relevance of the identified outliers and determine whether adjustments to the temporal filter are necessary. This iterative process ensures that the time bands are clinically meaningful and accurately capture the expected patterns of medical code distributions. Subsequently, all newly proposed temporal filters resulting from the adjustments are re-evaluated using machine learning functions. These machine learning functions analyze the updated data and provide insights into how well the new time bands align with statistical assumptions regarding the distribution of medical codes.

Once temporal filters are clinically determined, an iterative process may begin. This process involves capturing all patient encounters within a time period associated with each medical code. An automated analysis using a distributional slope technique with Benjamini-Hochberg false discovery rate (BH FDR) correction is then applied to determine which temporal filters require expansion. This analysis helps identify areas where the existing time bands may not adequately capture relevant data points.

For each medical code time band, all patient encounters are captured and an automated distributional slope analysis with a Benjamini-Hochberg False Discovery Rate Correction determines which time filter needs expansion. Clinical input is then used to determine the new time band and an additional distributional slope analysis (with BH FDR) determines the validity of the new time filter and the time filter is sent back if deemed not appropriate, until the clinically and statistically significant is derived. A machine learning model may be used to suggest the most appropriate temporal filter based on the distribution of the patient data. The machine learning model streamlines the decision-making process by leveraging computational analysis to identify optimal temporal filters for capturing relevant patient encounter information for determining the rating.

These filters may be important for correlating complications to procedures at an outpatient facility. For example, if a first person in a first demographic performed a type of procedure at an outpatient facility and experienced complications two weeks after the type of procedure, these complications may not be correlated to the type of procedure. However, if a second person in a second demographic performed the type of procedure at the outpatient facility and experienced complications two weeks after the type of procedure, these complications may be correlated to the type of procedure. This approach for using a variety of data sources to understand patterns, risk factors, and correlations related to different procedures and patient demographics strengthens the system's ability to evaluate outpatient facility metrics, including complication rates and treatment effectiveness, but also ensures that patient privacy and data security remain paramount throughout the process.

In some examples, machine learning functions (e.g., a machine learning model) may be used to populate gaps in the data sets relating to complications from outpatient facilities. Using data sources, such as, but not limited to databases (e.g., Lexis Nexis™) and insurance aggregations, low volume gaps identified during logistic regression analysis. Identified gaps may be routed to an AI agent workflow, which may use a large language model (LLM) agent find similar patient demographics and relevant medical codes (e.g., ICD10 or cpt codes) in secondary or tertiary claims tables. The LLM may be trained to parse through medical code and tertiary claims tables. Although such data may be accessed by a human, the sheer volume of such data and the formatting of the data (e.g., medical codes) would make such a task impossible to perform by a human. That is, a human cannot process and understand medical code data.

In some examples, machine learning methodologies may be used with new procedures (e.g., treatments) or rare procedures. In such examples, data used to understand patterns, risk factors, and correlations may be limited due to the rare or new nature of the procedure. Additionally, in some examples, outpatient encounter records may not document or include patient information such as past medical history, comorbidities, medication lists, or biometric data. Therefore, machine learning methodologies may be used to access other data sets to fill in gaps in patient information. For example, data set A contains data that is missing in data set B. And data set B contains other data that is missing in data set A. Combining these data sets results in less missing data than either data set on its own, thereby increasing overall data completeness and accuracy

This may enable a complete risk adjustment, tracking of complications, and quality measurement. Data mining and machine learning techniques may also be used to discover the complication patterns that exist in healthcare data and medical literature. This step identifies the association between medical treatments and procedures and medical complications that occur in the real world, whether or not a causal relationship has been established.

In some examples, missing data may also be inferred by the presence of data that indicates or infers the existence of earlier data. An example is a medical claim for the leasing of durable medical equipment, such as a hospital bed, because of a specific medical diagnosis. The presence of a specific medical diagnosis that is not in the original data set, may be inferred and retrospectively restored based on later medical claims. In such examples, the machine learning model may be trained to correlate the existence of one event, such as a medical diagnosis, based on the presence of another event, such as leasing of a specific medical device.

Systematically, filling gaps in medical history data produces more complete records that may enable complete risk adjustment by the inclusion of clinically relevant co-morbidities and risk factors. Along with more accurate tracking of complications, and quality measurement.

Data mining and machine learning techniques may also be used to discover novel complication patterns that exist in healthcare data and medical literature. This step identifies the association between medical treatments and procedures and medical complications that occur in the real world, whether or not a causal relationship has been established.

Some implementations involve the application of automated, dynamic temporal filters of variable intervals of time following a medical encounter or procedure for which a complication could be determined to be more likely than not to be casually related to a preceding medical encounter. Automated temporal filter (e.g., time band) generation may also undergo expert analysis and review. The output of each complication temporal filter may be monitored and audited by healthcare experts for statistical validity and stability. The analysis may be used to train the system to enable system learning, performance optimization, and discovery of new complications.

To determine complication rates, the computer system intakes medical health records and/or medical billing claims data for outpatient facilities. In one embodiment, the computer system intakes all Medicare patient medical records for those who undergo any procedures or treatments at the outpatient facilities within the Medicare network. For each patient, the data extracted, organized, and stored from the medical records includes, but is not limited to, comorbidities and the overall health condition of the patient prior to the outpatient procedure or treatment, the procedure or treatment performed, the outpatient facility performing the procedure or treatment, all complications that arose during the procedure or treatment regardless of perceived cause, all complications that arose during the patient's stay in the outpatient facilities regardless of perceived cause, and all complications arising over a given period post-procedure/treatment regardless of perceived cause. The timing of each complication vis-à-vis the date that the procedure/treatment was performed is also included as part of the data set that the computer system extracts from the medical records. Other data may also be included from the medical records based on the record set that is used to form the data set, e.g., identifying each facility as participating in Medicare.

The computer system may locate the data necessary for the determination of complication rates by scanning for certain codes commonly used in health records to refer to procedures and conditions. Examples of such codes include Current Procedural Terminology (“CPT®”) codes, International Classification of Diseases, Tenth Revision (“ICD-10”) codes, and Healthcare Common Procedure Coding System (“HCPCS”) codes, but other consistent coding systems may be used. Additionally, the necessary data may be pulled from health records and/or medical billing claims data based on the use of word searches, Boolean searches, native language searches, and other known methods for identifying specific data from mass record sets.

Once the computer system ingests the pertinent data, the system determines complication rates associated with a particular medical procedure or treatment using a process that parses complications to isolate and identify complications that most likely resulted from the procedure or treatment. The determination of complication rates involves at least the following actions. From the mass data from outpatient facilities, the computer system sorts the records by procedure or treatment. For example, records relating to all outpatient knee replacements are collected and grouped separately from other unrelated procedures, such as records relating to outpatient cardiac stent placement, cataract surgery, etc. For each procedure or treatment group, the computer system also identifies whether any pre-selected complications occurred during the procedure or treatment, during the stay at the outpatient facility, and after release from the outpatient facility. The identification of complications after release may be time limited, for example, to the review of records one year, two years, three years, four years, or five years following the procedure or treatment. The review period may vary according to procedure or treatment, e.g., the same health record may be searched for complications arising within three months following a tonsillectomy, within twelve months for treatment of a stroke, within two years following the implantation of pacemakers, etc. In some embodiments, the review period of the health record following a procedure or treatment may be dynamically updated as the computer system intakes more patient data regarding particular complications arising from a procedure or treatment.

When a pre-selected complication is identified in the health record of a patient, that complication is not automatically attributed to the procedure or treatment. Instead, the complication of interest is evaluated to determine if the complication is likely related to the procedure/treatment or not. For example, if a patient experienced a complication that could be related to the procedure or treatment, but that patient had pre-existing conditions or comorbidities that could also have caused the complication, the complication is not included in the calculation of the complication rate for the given procedure or treatment. A complication is further evaluated based on the timing of the complication using temporal parameters derived from one or more sources. If the timing of the complication falls within a certain time band, such as, from the time of the procedure or treatment through a specific range of time after the procedure or treatment, the complication is deemed related to the procedure or treatment and included in the complication rate. If the complication occurs outside the time band, the complication is not included in the calculation of the complication rate for the procedure or treatment. In the present disclosure, the time band may also be referred to as a temporal filter, hereinafter used interchangeably.

In some embodiments, a time band may be derived from the data collected from all outpatient facilities in a comparative cohort for a particular complication. Time bands can then be determined by aggregating information about the time between the procedure or treatment and the particular complication of interest. The computer system uses the distribution of time from procedure/treatment to complication to identify the average (or median) time during which a complication occurs for a given procedure or treatment. The time band is then derived from this distribution based on selected criteria. As an example, the time band may be plus or minus one standard deviation from the average time for the complication to occur post-procedure/treatment. As another example, the time band may be plus or minus two standard deviations from the average time for the complication to occur. The time band alternatively may be determined around the median time to complication, which may be particularly useful when the time to complication distribution does not fit a traditional bell curve. The process for determining time bands may involve a combination of data science and medical expert analysis.

In some examples, data mining and machine learning techniques may be used to discover the complication patterns that exist in healthcare data, medical literature, and/or other data sources. In such examples, an association between medical treatments and procedures and medical complications that occur in the real world may be identified, whether or not a causal relationship has been established. In some examples, the temporal filters may be dynamically adjusted as additional information is identified from data mining. These temporal filters may filter complications that are more likely to be causally related to a preceding medical encounter, aiding in the identification of potential adverse events. The automated temporal filter generation may undergo expert analysis and review to ensure accuracy and reliability.

As discussed, temporal filters may also be determined and/or updated from sources other than, or in addition to, information collected from the outpatient facilities. As non-limiting examples, information regarding particular complications may be obtained from literature. In some examples, a computer system may scrape the internet, scientific and/or clinical databases, journal collections, or publicly available clinical trial data. Such a scrape could include data from treatises, training materials, journal articles, case studies, and/or clinical studies. In some such examples, one or more data sources may be continuously monitored for new publications. When a new publication is identified, a machine learning model may analyze the publication to determine whether the publication includes information regarding a procedure, a patient demographic, complications, and/or other relevant information. The machine learning model may include a large language model (LLM) trained to read and analyze publications, such as PubMed, surgical registries, clinical trials, peer-reviewed medical journals, and medical reference texts. The data in the publication may be used to update existing data on patterns, risk factors, and/or correlations related to different procedures and patient demographics. The updated data may then be used by the machine learning model or temporal filter function to update the temporal filter for a specific procedure and/or patient demographic. In addition to continuously monitoring data sources for publications, the system may perform periodic searches of one or more data sources, such as the internet, for new publications. These searches may be automated internet searches.

In some examples, agentic workflows of LLM agents may be specified to gather, store, process, and evaluate new medical data. Agentic workflows refer to processes or workflows in which intelligent agents, such as LLMs, are actively involved in performing tasks, making decisions, and interacting with data or systems autonomously or semi-autonomously. In some examples, a research agent (e.g., AI agent) may gather new information from known sources such as Meta-Analyses, Systematic Reviews, Randomized Controlled Trials, and Cohort Studies, among others. The research agent may summarize and store this information. Another AI agent optimized for evaluation and trained on RAG (Retrieval Augmented Generation) with a prioritized hierarchy of trust and grading may assign valuations for each evaluated data object. The RAG may be further enhanced with Federal Drug Administration, Centers for Medicare and Medicaid Services, and Health and Human Services updates including black box warnings, contraindications, and new clinical indications, among others. A research designated agent trained on PubMed among other datasets, such as the Cochrane Library and web sources (e.g., ClinicalTrials.gov) may search and alert the organization to new topics or topics with a delta in frequency in current literature. A reporting agent may consolidate, evaluate, and surface gathered information for inclusion in clinical review based on the established trust hierarchy and RAG training.

As discussed, specialized agents may be trained to perform various tasks. In some examples, like research agents collect and store medical data from trusted sources, while specialized evaluation agents assess the value of these data elements using advanced models, such as RAG with regulatory updates. Another agent may monitor literature trends, and a reporting agent compiles and presents this information for clinical review, ensuring adherence to quality standards and updated insights from various sources.

In some examples, to implement the described scrapers, a comprehensive data collection framework is established to gather information from diverse sources such as, but not limited to, literature, scientific and clinical databases, journal collections, and public clinical trial data repositories. This framework includes setting up data scraping functions to extract relevant data from various online sources, such as, but not limited to, treatises, training materials, journal articles, case studies, and clinical studies. Additionally, integration with application program interfaces (APIs) provided by databases and clinical trial repositories enables direct access to structured data. Continuous monitoring of these data sources may be achieved through automated scripts and services that periodically check for new publications or updates. Change detection functions are used to trigger data retrieval processes whenever new information is detected.

As discussed, machine learning model may be specifically trained on publications and other data sources, such as medical publications, such that the machine learning model is trained to extract pertinent details such as procedures, patient demographics, complications, risk factors, and correlations. This model acts as the core engine for analyzing and processing the extracted data. A well-defined data processing pipeline is designed to handle the extracted information, performing tasks such as data cleaning, transformation, and integration with existing datasets. An update mechanism is implemented to ensure that the machine learning model and temporal filter function promptly incorporate newly processed data into the temporal filter for specific procedures and patient demographics.

The system may use an AI agent to perform guided evolution of genetic algorithms. These evolutions may optimize various solutions including lookback windows, demographic slicing, and machine learning algorithms such as Logistic Regression, XGBoost and others. This method of augmented evolving may also evaluate data beyond our defined cohorts and surface information for clinical review. The AI agent would play god in creating variations to test and to judge algorithms for survival and further use.

In some examples, automated searches may also be scheduled at regular intervals across data sources using keyword-based search algorithms to target relevant publications related to procedures, complications, patient demographics, and risk factors. That is, the automated search may be performed in addition to, or alternate from, the monitoring function. This ensures comprehensive coverage and timely updates of the system.

Integration and deployment of the system involve developing APIs and services to facilitate seamless communication between data sources, the machine learning model, and the temporal filter function. The system may be deployed on a scalable cloud infrastructure to handle varying loads and ensure high availability. Monitoring and logging mechanisms are put in place to track system performance, data updates, and address any potential issues promptly.

The dynamic updating of the temporal filter offers advantages in healthcare analytics by ensuring the system remains responsive to changing data patterns. This real-time adaptation capability enables the system to incorporate the latest trends and temporal variations in healthcare data, leading to more accurate and relevant insights. Moreover, the dynamic filter is adept at detecting emerging trends and patterns in healthcare data. As such, aspects of the present disclosure continuously learn from new information, allowing the temporal filters to adapt to early indicators of potential issues. This proactive approach helps healthcare providers stay ahead of evolving healthcare scenarios and make informed decisions promptly. Additionally, the system's enhanced predictive accuracy, driven by continuous learning and adaptation, contributes to improved patient safety. By promptly identifying potential adverse events or complications, healthcare providers can take preventive measures and optimize treatment plans for better patient outcomes.

In summary, the dynamic updating of the temporal filter ensures that the healthcare analytics system remains agile, responsive, and capable of providing timely and accurate insights. This adaptability is crucial for navigating the dynamic landscape of healthcare and making data-driven decisions that ultimately benefit patient care and safety.

The time bands may also be informed by expert analysis and review. As an example, time bands may be derived based on the experience and judgment of the experts alone or in combination with complication timing data from the outpatient facility records and/or from relevant literature. Additionally, such experts may be used to review and potentially adjust the time bands developed from the aggregation of outpatient facility data and/or literature. In that scenario, the expert may adjust the time band based on their credentials, experience, or other informed understandings of the procedure or treatment and resulting complications. Experts for time band determinations may be chosen based on any criteria, including but not limited to: medical certification; board membership; accreditation; degree(s) and/or training received; which schools the expert attended; which internships and/or fellowships the expert has undertaken; the hospitals for which the expert works or has worked, the number of years that the expert has practiced; the number of procedures or treatments performed; and/or other factors may identify leading practitioners in the field. In addition to, or instead of, using actual experts in the field to evaluate time bands, the computer system could be trained to mimic an expert's evaluation.

The use of literature and expert analysis (real or simulated) to develop time bands for complications may be especially helpful for new or rate procedures or treatments, where there may be insufficient data points from facilities to establish a standard or reliable distribution of the timing for the complication from health care records alone.

Time bands used by the computer system to determine complication rates may be static or may be updated periodically. As one example, if the applied time bands are determined based at least in part on the data obtained from outpatient facilities, such time bands may be continually and/or automatically adjusted as new data is collected. To do so, the new data may be added to the existing data of the comparative cohort, allowing for a new distribution of time from which a new time band may be derived and then applied to determine the complication rate.

Once the computer system has identified which complications in the data set are correlated to a procedure or treatment, the computer system can determine the average, or alternatively median, complication rate for a particular complication for a particular procedure or treatment. The average/median complication rate per procedure or treatment is identified for each member of the comparative cohort, as is the average/median complication rate per procedure or treatment for the comparative cohort as a whole.

Because the computer system stores the complication rate per complication per procedure or treatment for each of the members of the cohort as well as the rate for the cohort as a whole, the complication rate for a particular outpatient facility in the cohort can be compared to the complication rate for the entire cohort. Alternatively, the complication rate for a particular outpatient facility in the cohort can be compared to the complication rate for a subset of the entire cohort, for example, a cohort chosen based on geography, specialty, etc. Similarly, the complication rate for a selected group of facilities from a cohort can also be compared to the complication rates for the entire cohort or a subset thereof.

As an output of the comparison, the exact complication rate may be identified for the selected facility or group of facilities vis-à-vis the comparative cohort. For example, the comparative rate of the facility may be identified as, e.g., 15% compared to 25% overall. The complication rate also may be identified as a percentage above or below the average/median. For example, a facility could be identified as −30% if it is performing below the average/median by 30% or +10% for facilities outperforming the average/median by 10%.

The complication rate for an outpatient facility may be further identified using a scoring system. As only one example, the computer system may identify a facility's complication rate out of a five-star system where the number of stars depends on how its complication rate compares to the average/median of the comparative cohort (e.g., by the difference in percentage from the average/median, as measured by standard deviations, a set percentage difference, or a percentage difference that dynamically varies depending on the procedure). As another example, a facility may be identified as ranking between 1 to 10 based on a comparison to the average/median of the comparative cohort. Another form of score may be word based, where the word “scores” reflects the order of complication risk, such as, for example only, using the words “underperforming,” “average performance,” “overperforming” to reflect complication rates for a given facility.

As an output of the comparison complication rate and scoring system may be to schedule an appointment at the highest scoring or best fit medical facilities and providers via integration with online appointment scheduling (OAS) and online appointment request (OAR) platforms.

The computer system may also adjust complication rates and/or the scoring of such complication rates vis-à-vis the comparative cohort based on the health of the patient population undergoing the procedure or treatment at a particular facility. Even when comorbidities and/or pre-existing conditions are disregarded in the determination of whether a complication is related to a particular procedure or treatment, other factors relating to the patient population for the facility may affect outcomes. The computer system may thus include normalization factors that apply “handicaps” to facilities based on their patient population. As an example, the average age of patients undergoing a procedure or treatment at an outpatient facility may affect the complication rate, which should thus be taken into account when determining the complication rate or its relative scoring.

The complication rate data from the computer system may be searched and/or sorted in various ways to maximize its utility. For example, the system may be searched by procedure or treatment. The result of that search may be the average or mean complication rates for each of the most common complications associated with the procedure or treatment. As another example, a user may search for a particular complication regardless of the procedure or treatment. The result of that search may be the identification of procedures or treatments that give rise to that particular complication and how likely that complication will occur for each. As another example, the system may be searched by procedure/treatment and for a particular complication. The result of that search will provide the average or mean complication rate for the complication at interest for the procedure or treatment. As a further example, a user may search for a particular facility, or group of facilities, to identify complication rates at that facility for one or more procedures or treatments.

Additionally, the computer system further may be used to identify the total overall complication rate of a cohort of facilities for a given procedure or treatment by aggregating the complication rate for each tracked complication for that procedure or treatment. A total complication rate for all complications of all procedures/treatments for a particular facility or group of facilities may also be extracted by the computer system.

The computer system may also be capable of searches based on geography, specialty, distance from the user, the number of given procedures or treatments performed, or other criteria that may be of interest. Searching by multiple parameters is also contemplated. The user may access the computer system, e.g., via a webpage, website, portal, or via the download of software-enabled program.

The computer system has many uses that promote better health care results. In addition to providing for comparison of a given outpatient facility or group of facilities to the comparative cohort, the computer system may analyze the data set in other ways. In some examples, each of the facilities of the entire comparative cohort or a subset thereof may be ranked. Ranking could be, as non-limiting examples, by complication rate for all procedures or treatments, the complication rate for a particular procedure or treatment, the complication rate for a particular complication for a particular procedure or treatment, etc. This ranking may be used, as examples only, as a marketing tool by a facility, for community awards and recognition, as a factor in determining staff and provider bonuses, and/or to inform decisions regarding equipment, personnel, or training at the facility. The use of the computer system also may help a health care group, such as a parent company, determine how to allocate its budget among its related or affiliated facilities and/or assist in decisions to acquire, invest, or divest in a particular facility.

In addition, the computer system may be used to identify outpatient facilities that are performing below average, or alternatively, below a set expected performance level. Various actions may be taken for underperforming facilities by, e.g., the public, potential patients, physicians and other health care providers, facility administrators, regulatory bodies, certification bodies, and other interested parties. As only a few examples, the identification of a facility as underperforming may inform the choice of facility by patients, providers, and employees. It may also lead to further inspection, evaluation, and improvement of services, equipment, providers, funding, management, and the like at the facility. Identifying underperforming outpatient facilities may also lead to corrective action by facility management to address deficiencies and/or cause governmental, regulatory, or certification bodies to impose more stringent operational or reporting requirements, assess fines, and/or otherwise cause (or demand) improvement.

In a similar vein, outpatient facilities identified by the computer system as overperforming can, e.g., attract patients, providers, and employees; reward providers or staff and/or inform bonus decisions; have used as a marketing tool; lead to recognition, investments, and/or incentives for the facility; be used for benchmarking or standards for other facilities; and/or be tapped for training lower performing facilities.

As discussed, current analysis of procedures or treatments at outpatient facilities relies heavily on patient surveys, perceived reputational rankings of the facility, and other factors that are subjective and do not directly assess patient outcomes. Such analysis is unreliable and unlikely to provide a clear picture regarding the abilities of the outpatient facilities compared to the average outpatient facility performing the same procedure or treatment.

The absence of a reliable and reproducible methodology for evaluating outpatient facilities outcomes presents real world problems that affect several aspects of healthcare. It hampers the ability of health care providers and health care employees to compare and select appropriate facilities when they are deciding where to work, and/or obtain or maintain privileges. Having a system that permits useful comparative evaluations for outpatient facilities further allows the community, the facility owners, regulatory bodies, and the like to identify poorly performing facilities for audits, improvement plans, or closures. Alternatively, a computer system that allows for the identification facilities that are excelling may benefit from the expose, attracting better providers and employees, increased investment, or other advantages deserved by the outperforming facility.

An outpatient facility can also use a robust and reliable system of evaluating outpatient facilities to evaluate its own historical performance and determine if there is improvement or a decline in patient outcomes. Further, insurers may use the system to identify which outpatient facilities should be in-network, and the management of groups of affiliated facilities may use the system to identify outpatient facilities that should be acquired or divested.

There exists a need for a computer system and methodology that correctly identifies when complications are related to the procedures or treatments performed and evaluates complication rates relating to outpatient procedures/treatments in a quantifiable, reliable, verifiable, reproducible, transparent, and objective manner. Current evaluative systems may be based on subjective impressions of care that may be unrelated to health results, uninformed and unreliable conclusions by a patient or health care provider as to whether a complication is related to a procedure/treatment (which may result in underreporting or overreporting of relevant complications), and using limited data that fails to capture pre-existing conditions and comorbidities as well as complications that do not arise during the stay for the procedure or treatment. Various aspects of the present disclosure address these deficiencies.

Particular aspects of the subject matter described in this disclosure can be implemented to realize one or more of the following potential advantages. In some examples, applying a computer system to identify complications at outpatient facilities reduces recall bias, unreliable reporting of complications, improper correlation of complications to procedures, and addresses serious data gaps relating to the health of the patient. As another example, applying a computer system to identify complications from outpatient facilities may improve a user's ability to identify reliable outpatient facilities. Improving the user's ability to identify reliably outpatient facilities provides a better user experience while also reducing an amount of time needed to identify reliable outpatient facilities. Reducing the amount of time used to identify reliable outpatient facilities may reduce network bandwidth because a user spends less time searching for facilities.

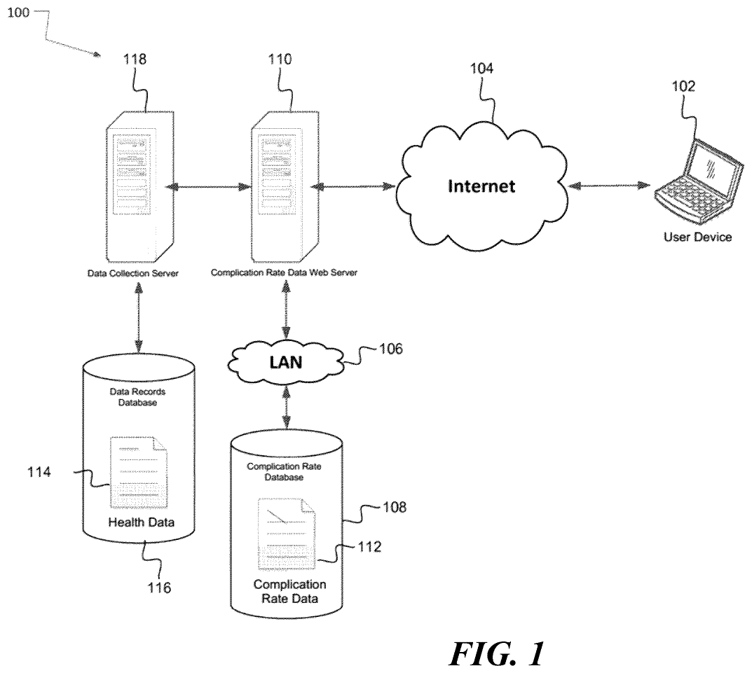

FIG. 1 illustrates a logical representation of a network environment 100 in which users are provided access to a database containing complication rate information, in accordance with various aspects of the present disclosure. As used herein, a “user” is any person using the network 100, typically one seeking complication rate information for outpatient facilities. In an embodiment, the network environment 100 includes a user device 102, a communications network 104, a local area network 106, a complication rate database 108 containing complication rate data 112, and a web server 110. Although only one user device 102 is shown, multiple user devices could communicate with web server 110. The user device 102 may be a desktop computer, a laptop computer, a mobile phone device, a tablet device, or any other device that may access the network 104 that gives access to websites or applications. In addition, the communications network 104 may be any type of network conventionally known to those skilled in the art. In accordance with an exemplary embodiment, the network may be the global network, e.g., the Internet or World Wide Web. It may also be a local area network or a wide area network. While the network may be any type of network conventionally known to those skilled in the art, the network is described in accordance with an exemplary embodiment as the “Internet.” As such, communications over the network occur according to one or more standard packet-based formats, e.g., HTTP, HTTPS, H.323, IP, Ethernet, and/or ATM. Further, while only one web server 110 is shown, more than one server computer or separate servers, e.g., a server farm, may be used in accordance with various aspects of the present disclosure.

In embodiments, the web server 110, database 108, and complication rate data 112 are maintained by the company offering access to complication rate searching, although not necessarily so. The network environment 100 is not limited to any particular implementation and instead embodies any computing environment upon which functionality of the environment may be practiced.

The network environment 100 further involves a backend computer system. The computer system includes, inter alia, a data collection server 118. The data collection server 118 maintains a health records database 116 that stores health data 114 from patients undergoing procedures at outpatient facilities. The health data 114 includes, inter alia, data relating to comorbidities and pre-existing conditions, the procedures performed, and complications arising during or after the procedure on a patient-by-patient basis. This data may be obtained, for example, from patient health records and/or medical billing claims data. Each device in the network environment 100 may include one or more memories and one or more processors. The one or more respective memories of each device may be coupled with the one or more respective processors.

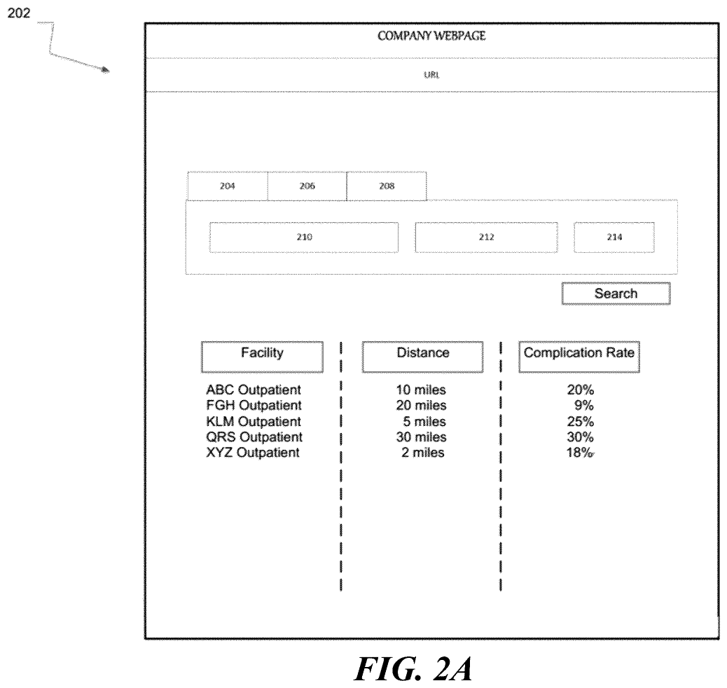

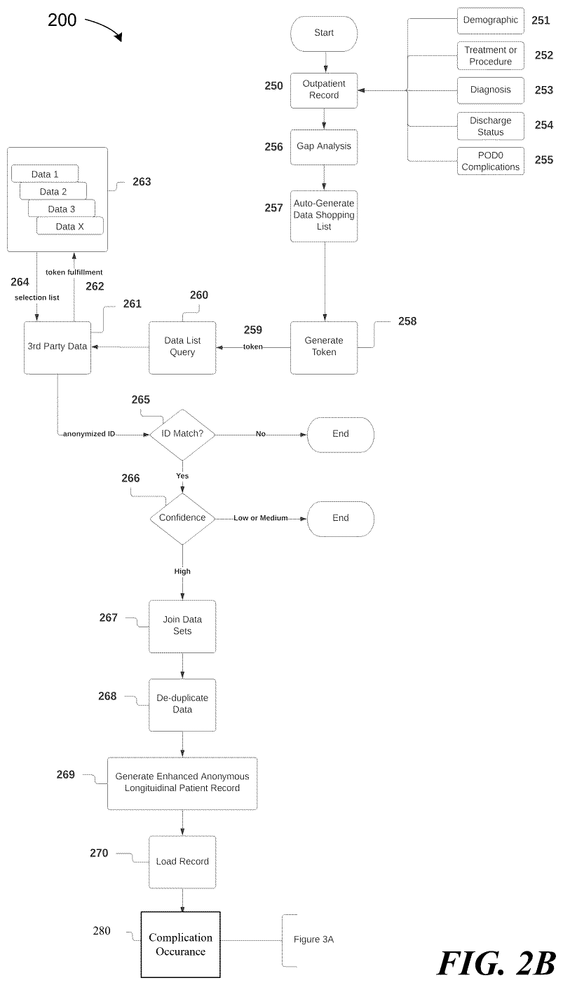

In accordance with an embodiment of the present application, a user may simply access the webpage 202 of the company hosting the complication rate data. The webpage 202 provides the ability to search and retrieve complication rate information 112 from the complications rate database 108, as shown in FIG. 1, to begin a search for complication rates. In such an embodiment, the user operates the user device 102 to access web server 110 over communications network 104 and receives the webpage 202 of FIG. 2A.

FIG. 2A is a schematic illustration of an exemplary webpage 202 for searching complication rates determined by the computer system 118, in accordance with various aspects of the present disclosure. The webpage 202 provides the user with the ability to search and retrieve complication rate information 112 from the complications rate database 108, as shown in FIG. 1, to begin a search for complication rates.

According to one embodiment of the present application, the user selects, from one or more search prompts, to undertake a particular search method. FIG. 2A displays search prompts 204, 206, and 208 for a user to select to search the complication rates. To return a complication rate, the user selects from one or more of the search prompts, where each search prompt, in turn, may provide textboxes to narrow the scope of the search.

As an example, in FIG. 2A, search prompt 204 may correspond to performing a search by procedure or treatment. After selecting search prompt 204, the user may type or select from a drop-down menu the specific procedure or treatment of interest in textbox 210, locations for facilities performing the procedure or treatment in textbox 212, and/or a particular complication in textbox 214.

As an alternative to, or in addition to, initiating a search by procedure or treatment using search prompt 204, the user could select search prompt 206 to search by facility. Textboxes corresponding to search prompt 206 (not shown) may permit the search for, e.g., a location for the facility (or a distance from the user); a specific practice area (such as cardiology), or a specific procedure or treatment (such as angioplasty). Another search method, which may be used alone or in combination with search prompt 204 and/or 206, is a search by, e.g., complication using search prompt 208. Textboxes corresponding to search prompt 208 (not shown) may permit the search for, e.g., a specific practice area (such as cardiology), a specific procedure or treatment (such as angioplasty), and/or a specific outpatient facility or set of facilities by location. Such a search using search prompt 208 may be helpful, for example, if the user is concerned generally about a particular complication due to a pre-existing condition, e.g., blood clots, heart attack, etc. Additionally, the search prompt 208 may be helpful if there are several procedures or treatments that may be chosen to address a particular health concern or condition.

After selecting the search criteria, the user may click on the “Search” icon to begin the search.

In other embodiments, a user may browse a search listing based upon, e.g., a procedure or treatment, a facility, or a complication. In such embodiments, no additional search criteria may be required to gain access to search listings.

In exemplary embodiments of webpage 202, a user typing within textbox 210, 212, and/or 214 invokes an autosuggest function. The autosuggest function organizes and displays search terms according to a probabilistic search algorithm. For example, if a user enters “implant” into textbox 210 to research physicians, various “implant” related results are suggested. The user may then highlight or select the desired search term from the autosuggest list. In other embodiments, a drop-down menu of various procedures or treatments may be displayed and selected by the user.

In some embodiments, the user may select a location for outpatient facilities. That location search may be by city, state, or zip code. Alternatively, the location search may be based on the distance from the user, for example, facilities within a distance radius values of, e.g., 1 mile, 5 miles, 10 miles, 25 miles, 50 miles, 100 miles, 200 miles, 300 miles, 400 miles, 500 miles, 1000 miles, and/or National.

Following the activation of the search by the user, results of the search can be displayed in a number of ways. An example of a table of results is set forth in FIG. 2A. In the example in FIG. 2A, a user has performed a search by procedure using search prompt 204. The user has specified a particular procedure in textbox 210, a location of the facility by distance from the user in textbox 212, and a specific complication of the procedure in textbox 214. The table of results in FIG. 2A thus provides the complication rate for the procedure, by distance, and by complication selected. In certain embodiments, the results of the search may be sorted by each column in the table, e.g., for FIG. 2A, the sorting may be by complication rate (low to high or high to low), by distance from the user (closest to furthermost or furthermost to closest), or by facility name (A to Z or Z to A).

FIG. 2B is a block diagram illustrating a process 200 for anonymizing and configuring outpatient encounter data, in accordance with aspects of the present disclosure. The process 200 may be performed, in all or in part, by one or more machines. For example, the process 200 may be performed by the user device 102, web server 110, and/or data collection server 118 described with respect to FIG. 1.

The process 200 begins at block 250 by receiving an outpatient record. The outpatient record may include patient data that is routinely collected during an outpatient encounter, or outpatient encounter data. The outpatient encounter data may include information from documentation received during a period between a procedure and a time of discharge if the discharge occurs on the same day as the procedure. Additionally, or alternatively, the outpatient encounter data may include information from documentation received within a time period after a procedure, such as twenty-four hours after a procedure. The outpatient encounter data may also include information regarding the patient's past medical history, which the process 200 may use to estimate risk.

In some examples, as shown in the example of FIG. 2B, the outpatient record 250 may include one or more of demographic information 251, treatment or procedure information 252, diagnosis information 253, discharge status information 254, or post operation day zero (POD0) complication information 255. The demographic information 251 may include, for example, the patient's age and sex at the time of the treatment or procedure. The treatment or procedure information 252 may include the treatment or procedure performed, such as a pacemaker implantation or an angioplasty. The diagnosis information 253 may include the diagnosis that is the reason for the treatment or procedure. The discharge status information 254 may include a description of the medical status of the patient at the time the patient left the facility and the location where the patient was discharged. For example, the discharge status information 254 may indicate that the patient is home in a stable condition, the patient expired, or that the patient is at a skilled nursing facility in a stable condition. The POD0 complication information 255 may indicate medical complications that occurred and were documented during the patient's outpatient encounter. The POD0 complication information 255 does not include complications that occurred after the patient left the facility.

At block 256, the process 200 performs a gap analysis. During the gap analysis, the process 200 performs an automated systematic analysis of the medical billing codes for each outpatient record. The process 200 may also classify the data type for the outpatient record information as demographic information 251, treatment or procedure information 252, diagnosis information 253, discharge status information 254, and/or POD0 complications information 255. Further, as part of the gap analysis, the process 200 may determine if the amount of data available in the outpatient record satisfies an information threshold.

In some examples, the gap analysis determines if patterns, risk factors, and/or correlations related to a procedure and patient demographic may be determined based on the outpatient record. The gap analysis may compare historical data from other outpatient records to the current data to determine if additional information is needed to accurately determine patterns, risk factors, and/or correlations related to a procedure and patient demographic. As discussed, such patterns, risk factors, and/or correlations may be used to determine a temporal filter. Additionally, as previously discussed, various gap filling techniques may be used to fill data gaps, such techniques may be employed in the gaps discussed with reference to the process 200.

In other examples, the gap analysis may be used to create a more complete patient record for determining if a complication occurred at an outpatient facility. If additional information is needed, the process 200 generates a list of supplemental data that may be available in other data sources to create a more complete longitudinal record. These data sources may be known sources, such as one or known data sources described above (e.g., medical record sources, insurance data sources, billing sources, etc.) The longitudinal record may include information from before, during, and after the outpatient encounter. At block 257, the process 200 auto-generates a data list specific for each patient. The data list may be referred to as a shopping list and includes supplemental data that is needed to cure gaps in the outpatient record.

At block 258, the process 200 generates an entity-specific encrypted patient-centric token 259. This token 259 replaces personally identifiable information (PII) typically used to identify protected health information (PHI). The tokenization process facilitates data exchanges without compromising the privacy or security of sensitive patient data. Essentially, the tokenization process involves transforming sensitive data into non-sensitive placeholder tokens, ensuring that only authorized entities can decipher and access the actual data.

At blocks 260-263, the generated token (259) is paired with the requester and patient identification information. This combined set of information is then transmitted to various third-party data sources, which can include entities like the Centers for Medicare and Medicaid Services (CMS), individual states' health departments, healthcare systems, individual medical facilities, third-party data aggregators, and/or other data sources. These third-party sources provide data relevant to the data shopping list generated at block 257.

Upon receiving the tokenized data along with the requester and patient identifiers, these third-party sources process the query and return a selection list (264). This selection list comprises data that matches the specific query made in block 257. Importantly, the selection list (264) contains data that has been processed and matched based on the encrypted tokens and identifiers, ensuring that sensitive patient information remains protected throughout the data exchange process.

At block 265, the process 200 determines if the data matching the patient identification was located and returned. If the data matching the patient identification was not located and returned, the process 200 may end. If the data matching the patient identification was located and returned, the process 200 may continue to block 266. At block 266, the confidence of the match is assessed. If the confidence of the match is low or medium, the process 200 may end. If the confidence of the match is high, the process 200 may continue to block 267.

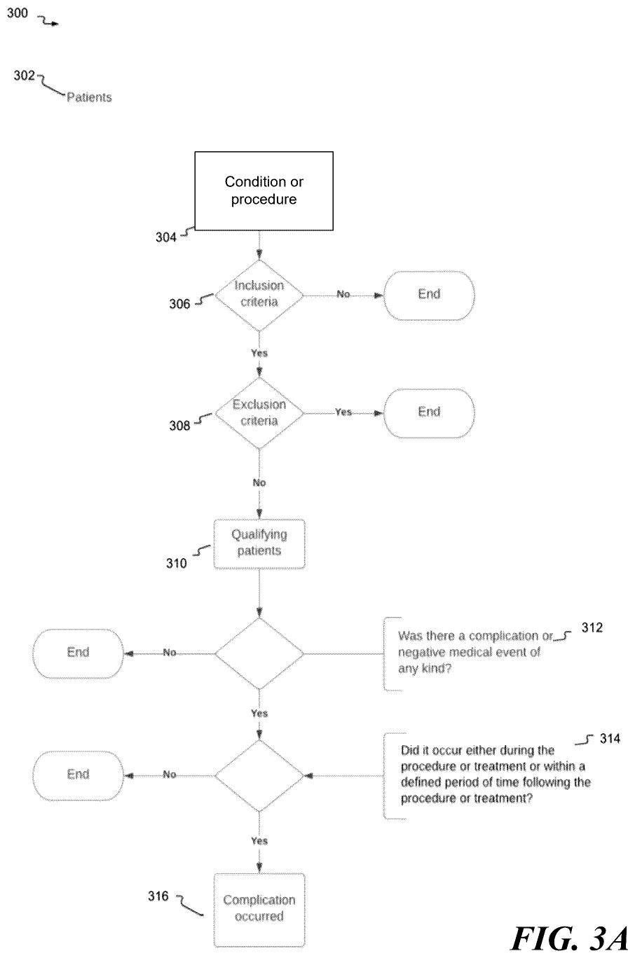

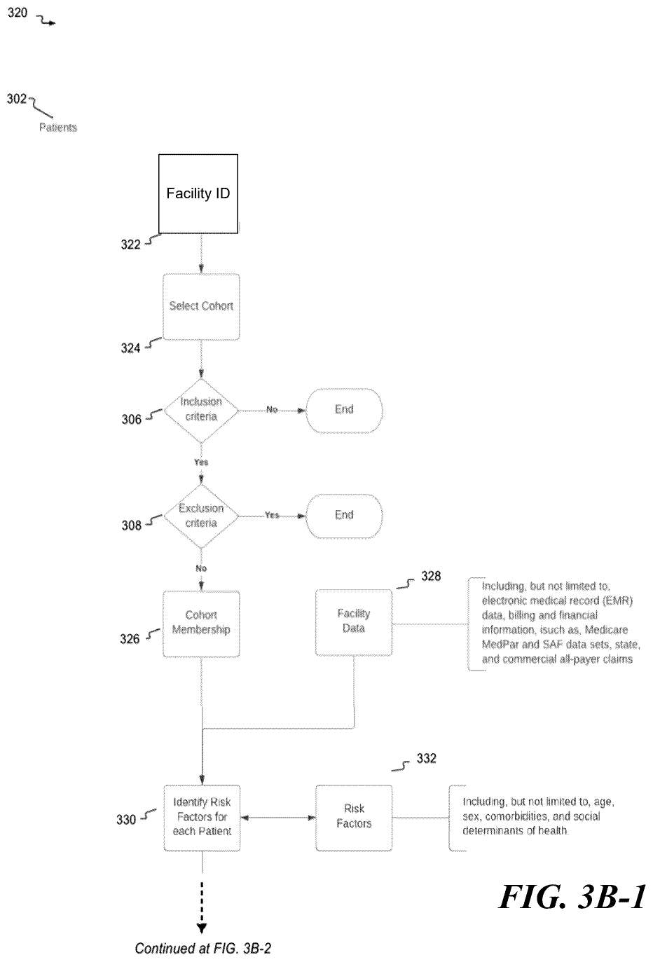

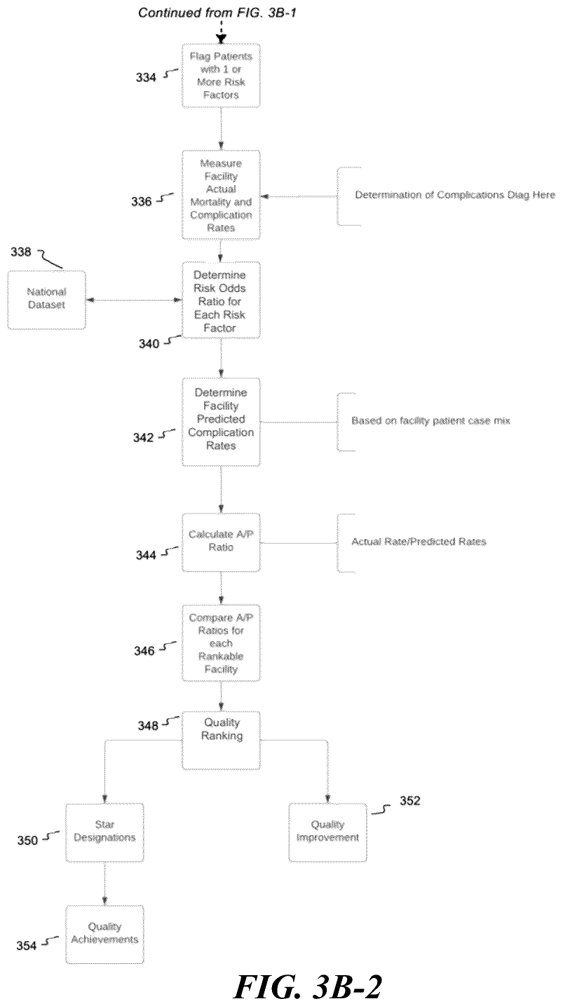

At block 267, the process 200 joins the outpatient record and third-party data into a single record. The process 200 may then remove duplicative data at block 268. Then, at block 269, the process 200 generates an enhanced anonymous longitudinal patient record. At block 270, the record is loaded into a ratings application. The ratings application may implement techniques described with respect to this disclosure to generate ratings. At block 280 the process 200 may determine whether a complication occurred within a time band (e.g., temporal filter) described with respect to FIG. 3A.

FIG. 3A is a block diagram illustrating an example of the analysis performed for including a particular complication into the complication rate determination, in accordance with aspects of the present disclosure. The identification of includable complications 300 includes the review of health data from patients 302. The health data, which may be from medical records and/or medical billing claims data or the like, is reviewed to determine whether the patient was treated for a particular condition or underwent a particular procedure or treatment, 304. There are often many different types of procedures or treatments that are highly related. Inclusion criteria 306 is applied to identify all of the like/similar procedures or treatments and group them together for purposes of determining the complication rate. As an example, inclusion criteria 306 could be a list of medical codes associated with similar or similar procedures or treatments.

Once all like/similar procedures or treatments are grouped, exclusion criteria 308 is applied to eliminate patients who have comorbidities or procedure/treatment deviations that greatly distinguish them from most patients undergoing the procedure/treatment. As one non-limiting example, patients that are replacing both knees at the same time may be excluded in the analysis of complication rates for knee replacement surgeries because the risk profile is very different for such patients. As another example, patients undergoing treatment for heart failure who also have metastatic cancer may be excluded; accrediting any particular complication to the treatment rather than to the cancer would be exceedingly difficult given the diverse complications that may arise from metastatic cancer.

Once inclusion and exclusion criteria are applied, a pool of qualifying patients 310 who underwent the procedures or treatments of interest remains. The next step is to identify patients in that pool who experienced a complication or negative medical event of any kind during or after the procedure or treatment, 312. If a complication occurred, and that complication is one being tracked for that particular procedure or treatment, a second review is taken where the timing of the complication vis-à-vis the procedure/treatment is determined, 314. If the complication occurred within the time bands established for that particular complication, the complication is counted as having occurred due to the procedure/treatment, 316. That is, complications are filtered based on the time bands (e.g., temporal filters) to increase the accuracy of the complication rate. Such complications are then used to determine complication rates.

FIG. 3B is a block diagram illustrating a risk adjustment process 320 used to score a facility based on its complication rates, in accordance with aspects of the present disclosure. FIG. 3B diagram spans two pages. On the first page, FIG. 3B-1, data from patients 302 is sorted by each outpatient facility for analysis, 322 (FacilityID). A comparative cohort of facilities is then selected, 324, among the facilities identified. For example, selected cohorts 324 may be all outpatient facilities in the U.S., all outpatient facilities in a particular state or region, all outpatient facilities treating a certain demographic (children, the elderly), all outpatient facilities in a particular insurance network, etc. From the selected cohort 324, a particular procedure or treatment is reviewed.

As was the case in FIG. 3A, like/similar procedures or treatments are grouped using inclusion criteria 306 and exclusion criteria 308 are applied to eliminate patients who have comorbidities or procedure/treatment deviations that would confound the complication determination. Facilities that remain after application of the inclusion and exclusion criteria are included as members of the comparative cohort, 326. For each facility in the cohort, facility data 328 is reviewed to identify risk factors for each patient, 330. Risk factors 332 may include, for example, comorbidities, age, gender, etc.

In some embodiments, the computer system uses logistic regression to determine which of the potential risk factors are statistically significant in predicting the outcome measure, e.g., a complication. The final model includes the risks factors that are statistically significant in predicting the clinical outcome (alpha=0.05) and may also include other risk factors that are identified by statistical and/or clinical expert review.

FIG. 3B continues on page FIG. 3B-2. In the example shown, patients that have one or more risk factors are flagged, 334. In step 336, the actual complication rate for a facility is measured, a determination that may be made, inter alia, using the complication logic set forth in FIG. 3A. Risk odds ratios, 340, are determined for each risk factor included in the model. An odds ratio is a way to express the impact of each risk factor. As one non-limiting example, having diabetes will increase the risk of a knee replacement complication by a factor of 2.1. Determining risk odds ratio are informed by information in the national dataset 338, e.g., which includes data from all patients in all outpatient facilities regardless of the selected facility cohort.

The predicted complication rate for a facility, 342, is determined by the summation of the individual patient record predicted values determined from logistic regression. The actual facilities complication rate 336 is then compared to the predicted complication rate 342 to develop the A/P ratio 344. In some embodiments, a test is conducted to determine if the difference between the predicted and actual complication rates is statistically significant. This test helps ensure that differences in the rates are unlikely to be caused by chance alone.

Relative quality rankings 348 for each facility in the selected facility cohort may be made using the derived complication rates. A quality ranking may be used by facilities to take quality improvement measures 352, such as obtaining consulting to identify areas for improvement and investigating possible root causes. Tracking rankings after implementing quality improvement measures can then be used to identify the impact of such measures.

Rankings can also be organized into various scoring methods. As one example and as shown in FIG. 3B-2, facility rankings may be translated into star designations 350. In one non-limiting example using a five-star scoring system, a facility may receive one star if its complication is higher than 1.645 standard deviations above the mean complication rate for the facility cohort. In the same scoring system, a facility that has a complication rate lower than 1.645 standard deviations from the mean may receive five stars. Correspondingly, facilities that fall within 1.645 standard deviations above or below the mean may receive three stars. Quality rankings may also be used to identify quality achievements 354, such as creating “best of” listings of facilities and the like.

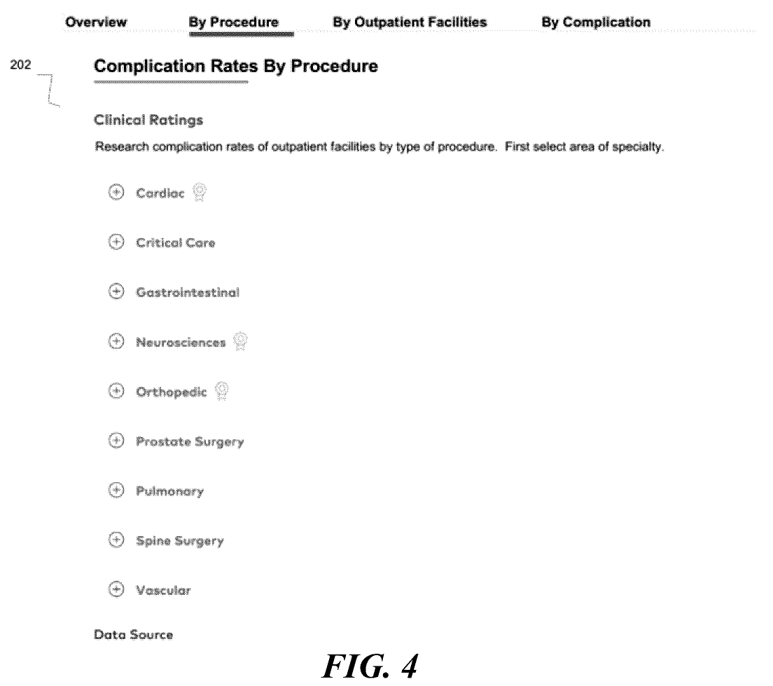

FIG. 4 illustrates an example of a search page used to identify complication rates by procedure or treatment performed, in accordance with various aspects of the present disclosure. Other options include searching by outpatient facility or by complication, though other search methods could be used. In FIG. 4, a list of medical specialties is provided from which the user can select procedures or treatments falling within that specialty. For example, procedures of a cardiac nature are combined under the label “Cardiac,” treatments relating to critical care are combined under the label “Critical Care,” etc.

In FIG. 4, beside each medical area there is a plus sign that, when selected, opens up a menu of procedures/treatments relating to that medical area. For example, upon selecting the plus sign for cardiac care, all procedures and treatments that have been tracked for complication rates automatically appear. Such cardiac procedures/treatments may include, as examples only, pacemaker implantation, angioplasty, etc. Alternatively, the menu to select procedures or treatments may contain a textbox that the user can populate with the procedure of interest.

At the bottom of FIG. 4, there is a link to the Data Source used by the computer system to generate the complication rate data. If the user selects the Data Source link, the link may contain information about the underlying data, such as where the information was obtained, the information used to determine complication rate, the criteria for including a given outpatient facility, etc. Including a Data Source link is optional and designed solely to provide background information that may be of interest to the user.

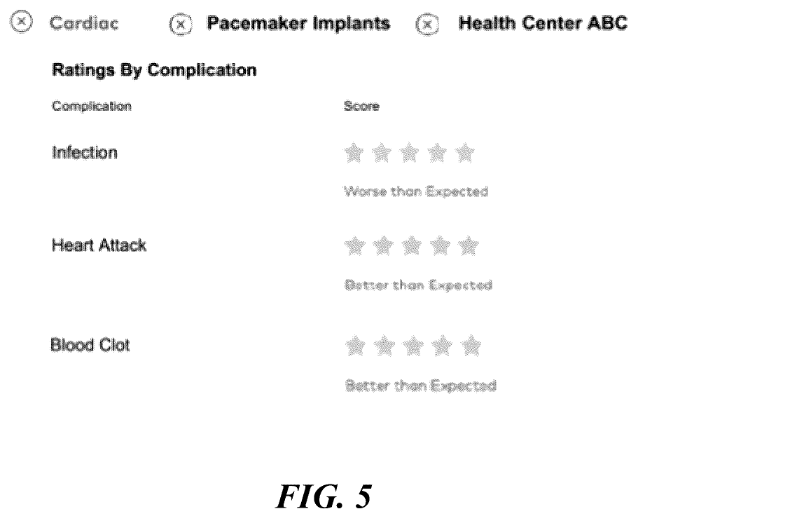

FIG. 5 illustrates an example of scoring that may be used to reflect complication rates for certain complications, in accordance with aspects of the present disclosure. As indicated at the top of FIG. 5, the user has selected the medical area for a procedure (Cardiac), a particular procedure (Pacemaker Implant), and a chosen outpatient facility (the Health Center ABC). Using the methodology described above, the computer system determines the complication rate of each tracked complication relating to the specified procedure at the specified facility and a scoring is provided for each complication. The complications in the example in FIG. 5 relating to implantation of a pacemaker are infection, heart attack, and blood clot. Other complications may be tracked based on the patient data obtained and medical understanding of complications that may be related to the procedure or treatment.

In FIG. 5, the scoring is based on a star rating system, where scores vary from one to five stars. Health Center ABC is scored based on the complication rate after pacemaker implantation for infection, heart attack, and blood clots compared to the average or median complication rate for all outpatient facilities included in the data set. In FIG. 5, one star has been awarded with respect to the infection complication, which indicates a complication rate of infection exceeding the average or median complication Alternative scoring systems may be used, such as a score out of three stars or four stars, a score of 1 to 5 or 1 to 10 or the like, or the use of words such as “underperforming, average performance, overperforming” or similar words reflecting an order of complication risk. As other alternatives, the computer system output may be the exact complication rate for a facility or the difference between the complication rate for a facility compared to an average or median complication rate of the facilities included in the data set.

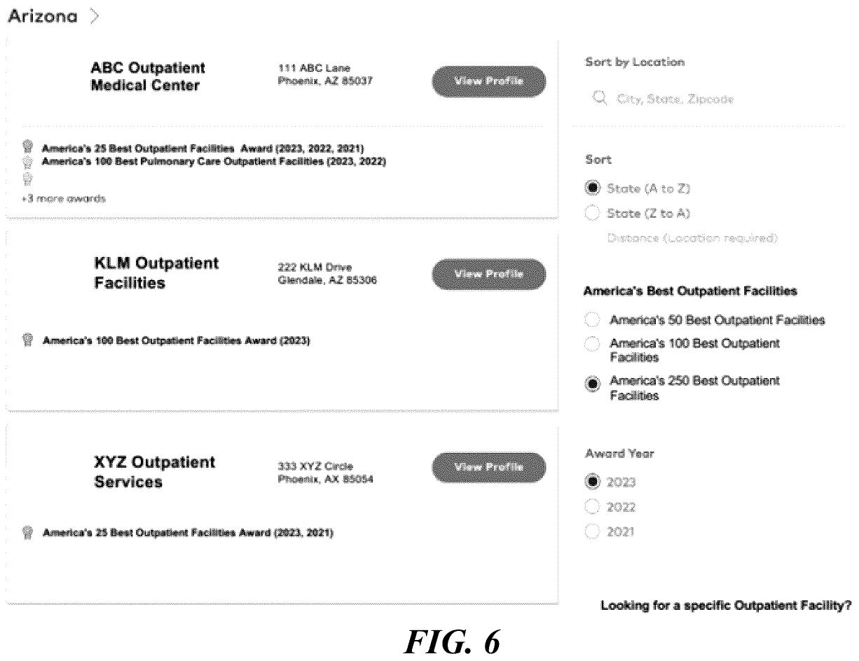

FIG. 6 illustrates a results screen where one criteria for search is for outpatient facilities, in accordance with various aspects of the present disclosure. In the example of FIG. 6, the outpatient facilities are ranked as one of the 50 best facilities, 100 best facilities, or 250 best facilities located in America. The “best of” lists may be based on, e.g., the overall complication rate for all procedures or treatments at the facility, the overall complication rate for all procedures or treatments within a particular medical area, the overall complication rate for a specific procedure or treatment, the rate of a particular complication among one procedure or treatment, multiple selected procedures or treatments, all procedures or treatments performed at the facility, etc.

In FIG. 6, another criteria for searching is the location of facilities by specifying a particular city, state, or zip code. Sorting of results may also be specified as by state from A to Z, by state from Z to A, or by the distance from a particular location. FIG. 6 also provides for the selection of the year in which the facility was ranked as a “best of” facility. Such yearly ratings for 2022 and 2021 do not exist, but they are included as a hypothetical example of how to search by a “best of” list.

The results in FIG. 6 correspond to search selections by the user for sorting by States (A to Z), facilities are within America's 250 Best Outpatient facilities, and Award Year. The exemplary results of this search are provided in an alphabetic scrolling list of facilities meeting the selected criteria. In FIG. 6, the user has scrolled to the state of Arizona.

In some examples, as shown in FIG. 6, a listing of facilities meeting the search criteria is provided. In this example, the facility address is provided, as well as a list of awards that the facility has received and the year of the award. The results also include a link by which the user may choose to view the “profile” for a facility. This profile could contain any information relating to the facility, such as a link to the website, etc.

At the bottom of FIG. 6, there is a link entitled “Looking for a specific Outpatient Facility.” This link may be used, e.g., to bring the user to an alternative search query website where the user may specify a particular facility or a group of facilities where the user wants to review complication rate data.

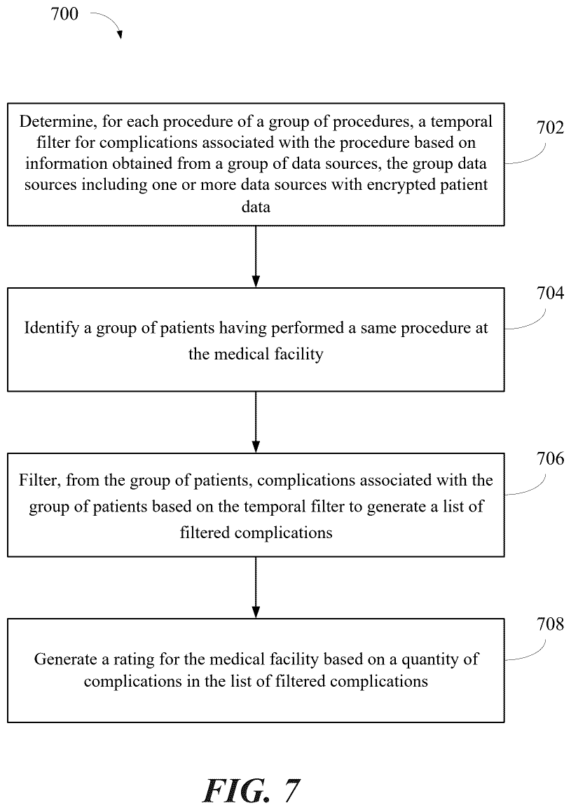

FIG. 7 is a flow diagram illustrating an example of a process 700 for filtering complications in order to determine a number of complications associated with an outpatient facilitate, in accordance with various aspects of the present disclosure. The process 200 may be performed, in all or in part, by one or more machines. For example, the process 200 may be performed by the user device 102, web server 110, and/or data collection server 118 described with respect to FIG. 1.

As shown in the example of FIG. 7, the process 700 begins at block 702 by determining, for each procedure of a group of procedures, a temporal filter for complications associated with the procedure based on information obtained from a group of data sources, the group data sources including one or more data sources with encrypted patient data. In some examples, the temporal filter may be determined, via a first machine learning mode, the temporal filter for the procedure by correlating patterns of complications to the procedure. The temporal filter may also be associated with a patient demographic.

The group of data sources include, but are not limited to, one or more of one or more internet sources, one or more scientific databases, one or more clinical databases, one or more journals, clinical trial data, one or more treatises, one or more training materials, one or more journal articles, one or more case studies, one or more clinical studies, studies patient health records, one or more medical billings claims data, and one or more expert statements. At block 704, the process 700 identifies a group of patients having performed a same procedure at the medical facility. In some examples, the medical facility is an outpatient facility. In some examples, identifying the group of patients includes receiving an outpatient record, identifying one or more data gaps in the outpatient record, identifying one or more data sources corresponding to the data gaps, generating a list of data for filing the one or more data gaps, generating a patient specific token, transmitting the patient specific token and the list of data to the one or more data sources, receiving data items corresponding to the list of data in according with transmitting the patient specific token and the list of data, removing duplicate data from the data items, joining the data items and the outpatient record. Additionally, in some examples, the outpatient record includes one or more of patient demographic information, treatment or procedure information, diagnosis information, discharge status, and zero day complications.

In some examples, the process 700 continuously monitoring one or more data sources of the group of data sources for an update. Additionally, the process 700 may review, via a second machine learning model, the update to determine whether the update includes additional information regarding the procedure, the patient demographic, and/or the complications. The second machine learning model may be an LLM. Additionally, the process 700 may updating, via the first machine learning model, the temporal filter based on the update including additional information regarding the procedure, the patient demographic, and/or the complications.

At block 706, the process 700 filters, from the group of patients, complications associated with the group of patients based on the temporal filter to generate a list of filtered complications. At block 708, the process 700 generates a rating for the medical facility based on a quantity of complications in the list of filtered complications. In some examples, based on the rating, a system may autonomously refer patients to the medical facility based on the rating. For example, if the rating is greater than a threshold, a referral system may refer patients to the medical facility.

In some embodiments of the present application, the computer system involves a backend computer system that is a data collection server. The computer system may receive the patient data (e.g., health record information and/or medical billing claims data) by accessing a web server over a communications network and receiving the webpage in any downloadable form. The computer system also may receive the health care record information directly from computer readable media, such as by one or more hard-drives or other data storage devices populated with the health records.

The computer system of the present application may develop complication rates from the patient health records through a computer process, and executable program, or from an article of manufacture such as a computer program product or computer-readable media. The computer program product may be a computer storage media that is readable by a computer system and encodes a computer program of instructions for executing a computer process that develops the complication rate data of the present invention.

After the patient data has been incorporated and the complication rates developed as outlined above, the computer system may maintain this information in one or more databases. For example, raw data may be stored together or separately from the complication rates determined from the raw data.

A user may access the complication rates developed by the computer system in various ways. As an example, the user may access or search the data from the computer system using a device that accesses the database(s) of the computer system through a communications network or a local area network. The user device may be any type of device with a network connection, including, but not limited to, a personal computer, a smartphone, a tablet, or another type of network device.

In one embodiment of the present application, the user may access or search the data from the computer system located on a web server. A user may also access or search the data of the computer system indirectly, using a copy of the information that has been gathered and recorded from the computer system. Such a copy may be in the form of a disc, thumb drive, hard-drive, or other data storage device, or in an electronic form that can be downloaded from the web.

In some examples, a computer system may be used to identify a complication rate for one or more complications related to a procedure or treatment performed at one or more outpatient facilities. The computer system may include a user device, a communications network, and a complication rate data web server. Additionally, the computer system may include a backend computer system in communication with the complication rate data web server. The computer system may also store one or more types of health data obtained from patients who have undergone the procedure or treatment at the one or more outpatient facilities.

The backend computer system may identify, from the health data, the complication rate for each complication related to the procedure or treatment at each of the outpatient facilities. Further, a complication rate data webserver may transmit, to the user device, one or more complication rates in response to a prompt received from the user device. The one or more complication rates may be for one or more complications related to the procedure or treatment at one or more of the outpatient facilities.

The health data may be obtained from one or more sources. For instance, the health data may be obtained from electronic medical records or medical billing claim data. Additionally, the health data may be obtained from Medicare electronic medical records or medical billing claim data. The health data may also be obtained from insurer electronic medical records or medical billings claim data.

The health data from each patient may further include risk factors. To determine which of the risk factors are statistically significant in predicting whether a complication will occur for the procedure or treatment, the computer system may use logistic regression. The computer system may further determine the risk odd ratios for each risk factor identified as statistically significant.

The health data for each patient may also comprise comorbidity data and the overall health condition of the patient prior to the procedure or treatment. The health data may additionally comprise all complications experienced by the patient that occurred during or after the procedure or treatment over a given period of time. The health data may further include the time that each complication arose with respect to the date of the procedure or treatment.

In some implementations, the computer system determines, from the health data, the actual complication rate for one or more complications. The complications may be related to the procedure or treatment performed at each of the outpatient facilities belonging to a cohort of outpatient facilities. The computer system may determine the actual complication rate by identifying the total number of procedures or treatments performed at the cohort of outpatient facilities. The computer system may also identify, for the procedure or treatment, one or more specific complications from which to determine a complication rate. The computer system may further determine which patients at each outpatient facility experienced one or more specific complications after undergoing the procedure or treatment at the outpatient facility and when each specific complication occurred following the procedure or treatment.

To determine the actual complication rate, the computer system may still further compare when the specific complication occurred to a time band for the procedure or treatment. The time band may be a pre-determined period of time during which the specific complication is likely to occur following the procedure or treatment. The computer system may also designate the specific complication as related to the procedure or treatment only if the specific complication occurs during the time band for said complication. The computer system may then derive the actual complication rate at each outpatient facility for each specific complication following the procedure or treatment by comparing the number of times the specific complication was related to the procedure or treatment to the total number of the procedures or treatments performed at the outpatient facility.

To determine the actual complication rate, the computer system may also exclude the health data from patients with comorbidities and/or the health data from patients who underwent a deviation from the procedure or treatment that significantly affects the complication rate for the procedure or treatment. The computer system may further determine the actual complication rate for the cohort of outpatient facilities as a whole for each of the specific complications related to the procedure or treatment. For instance, the computer system may average the actual complication rate for each of the outpatient facilities belonging to the cohort for each of the specific complications related to the procedure or treatment.

In some implementations, the computer system determines the predicted complication rate for one or more complications. The complications may be related to the procedure or treatment performed at each of the outpatient facilities belonging to a cohort of outpatient facilities. The computer system may determine the predicted complication rate by identifying the total number of procedures or treatments performed at the cohort of outpatient facilities. The computer system may also identify, for the procedure or treatment, one or more specific complications from which to determine a complication rate. The computer system may further determine which patients at each outpatient facility experienced one or more specific complications after undergoing the procedure or treatment at the outpatient facility and when each specific complication occurred following the procedure or treatment.

To determine the predicted complication rate, the computer system may still further compare when the specific complication occurred to a time band for the procedure or treatment. The time band may be a pre-determined period of time during which the specific complication is likely to occur following the procedure or treatment. The computer system may also designate the specific complication as related to the procedure or treatment only if the specific complication occurs during the time band for said complication. The computer system may further adjust the designation of a specific complication as related to the procedure or treatment. The adjustment may be based on the number of patients at the outpatient facility having statistically significant risk factors and the risk odd ratios for such statistically significant risk factors. The computer system may then derive the predicted complication rate at each outpatient facility for each specific complication following the procedure or treatment by comparing the number of times the specific complication was related to the procedure or treatment to the total number of procedures or treatments performed at the outpatient facility.

The computer system may also compare the actual complication rate for a specific complication related to the procedure or treatment for each outpatient facility to the actual complication rate for the specific complication for the cohort of outpatient facilities as a whole. The computer system may then assign a score for each of the specific complications related to the procedure or treatment to each outpatient facility based on the comparison. The computer system may additionally list each outpatient facility in the cohort of outpatient facilities in order of the actual complication rate from lowest to highest actual complication rate or highest to lowest actual complication rate.

In some implementations, the time band for the specific complication is a range of time determined by one or more measures. A first measure may be the average or mean time between the procedure or treatment and the occurrence of the particular complication for all of the outpatient facilities of the cohort. The range of the time band determined by the first measure may be based on a distribution of the time between the procedure or treatment and the occurrence of the particular complication for all of the outpatient facilities in the cohort. Additionally, the time band for the specific complication determined by the first measure may be adjusted by input from one or more experts in the field of the procedure or treatment.

A second measure may be information regarding the timing of the specific complication for the procedure or treatment that the computer system scrapes from the internet, databases, and/or other electronic sources of information regarding the timing of the specific complication for the procedure or treatment. The time band for the specific complication determined by the first measure and/or the second measure may be adjusted by input from one or more experts in the field of the procedure or treatment relating to one or both of the measures. Additionally, a third measure may be input from one or more experts in the field of the procedure or treatment regarding the timing of the specific complication for the procedure or treatment.

After the computer system determines the actual complication rate, the computer system may compare the actual complication rate for each outpatient facility of the cohort of outpatient facilities to the predicted complication rate. The actual complication rate and predicted complication rate may each be for a specific complication related to the procedure or treatment performed at each outpatient facility. The computer system may further identify, for each outpatient facility, the ratio of the actual complication rate to the predicted complication rate.

To further determine the predicted complication rate and the actual complication rate, the computer system may average the predicted complication rate and the actual complication rate for each of the outpatient facilities belonging to the cohort for each of the specific complications related to the procedure or treatment. The predicted complication rate and the actual complication rate in the determination may be for the cohort of outpatient facilities as a whole for each of the specific complications related to the procedure or treatment. The computer system may then compare, for the cohort of outpatient facilities as a whole, the actual complication rate to the predicted complication rate. Both the actual complication rate and the predicted complication rate may each be for a specific complication related to the procedure or treatment performed at all of the outpatient facilities in the cohort. Further, the computer system may identify the ratio of the actual complication rate to the predicted complication rate for the outpatient facility as a whole.

The computer system may additionally compare, for each complication related to the procedure or treatment, the ratio of the actual complication rate to the predicted complication rate for each of the outpatient facilities to the ratio of the actual complication rate to the predicted complication rate for the outpatient facilities as a whole. Then, the computer system may assign a score for each of the specific complications related to the procedure or treatment to each outpatient facility based on the comparison. Before or after the comparison, the computer system may list each outpatient facility in the cohort of outpatient facilities in order of the actual complication rate from lowest to highest actual complication rate or highest to lowest actual complication rate.

The computer system may be implemented to perform various techniques. In one technique, the computer system may be implemented to identify outpatient facilities that are in need of improvement. For example, a computer system may identify facilities having a higher complication rate for a selected complication than the corresponding complication rate for the cohort of outpatient facilities as a whole. The computer system may additionally be implemented to identify outpatient facilities performing the procedure or treatment with a lower complication rate for a selected complication than the complication rate for the cohort of outpatient facilities as a whole. Similarly, the computer system may additionally be implemented to identify outpatient facilities performing the procedure or treatment with a lower complication rate for a selected complication than the corresponding complication rate for the cohort of outpatient facilities as a whole.

In general, the embodiments of the present application relate to identifying the complication rate for one or more complications related to a procedure or treatment performed at one or more outpatient facilities. Each embodiment comprises a computer system comprising a user device; a communications network; a complication rate data web server; a backend computer system in communication with the complication rate data web server; and one or more types of health data obtained from patients who have undergone the procedure or treatment at the one or more outpatient facilities. In embodiments, the backend computer system of the computer system identifies from the health data the complication rate for each complication related to the procedure or treatment at each of the outpatient facilities. In embodiments, one or more complication rates for one or more complications related to the procedure or treatment at one or more of the outpatient facilities may be transmitted to the user device via the complication rate data webserver. The complication rate(s) to be transmitted may, in some embodiments, correspond to a prompt received from the user device.

In one non-limiting embodiment, the computer system may use health data obtained from electronic medical records or medical billings claim data. In some embodiments, such health data is obtained from Medicare electronic medical records, Medicare medical billings claim data, insurer electronic medical records, and/or insurer medical billing claims.

In non-limiting embodiments, the health data obtained for each patient comprises at least comorbidity data, the overall health condition of the patient prior to the procedure or treatment, all complications experienced by the patient that occurred during or after the procedure or treatment over a given period of time, and the time that each complication arose with respect to the date of the procedure or treatment. In other embodiments, the health data further includes, inter alia, risk factors for each patient.

In some embodiments, the computer system uses logistic regression to determine which of the risk factors are statistically significant in predicting whether a complication will occur for the procedure or treatment and/or identifies the risk odd ratios for each risk factor identified as statistically significant.

In some embodiments, the computer system determines from the health data the actual complication rate for one or more complications related to the procedure or treatment performed at each of the outpatient facilities belonging to a cohort of outpatient facilities. The computer system determines the actual complication rate by: identifying the total number of the procedures or treatments performed at the cohort of outpatient facilities; identifying for the procedure or treatment one or more specific complications from which to determine a complication rate; determining which patients at each outpatient facility experienced the one or more specific complications after undergoing the procedure or treatment at the outpatient facility and when each specific complication occurred following the procedure or treatment; comparing when the specific complication occurred to a time band for the procedure or treatment, wherein the time band is a pre-determined period of time during which the specific complication is likely to occur following the procedure or treatment; designating the specific complication as related to the procedure or treatment only if the specific complication occurs during the time band for said complication; and deriving the actual complication rate at each outpatient facility for each specific complication following the procedure or treatment by comparing the number of times the specific complication was related to the procedure or treatment to the total number of the procedures or treatments performed at the outpatient facility.

In another embodiment, the computer system determines from the health data the predicted complication rate for one or more complications related to the procedure or treatment performed at each of the outpatient facilities belonging to a cohort of outpatient facilities. The computer system determines the predicted complication rate by: identifying the total number of the procedures or treatments performed at the cohort of outpatient facilities; identifying for the procedure or treatment one or more specific complications from which to determine a complication rate; determining which patients at each outpatient facility experienced the one or more specific complications after undergoing the procedure or treatment at the outpatient facility and when each specific complication occurred following the procedure or treatment; comparing when the specific complication occurred to a time band for the procedure or treatment, wherein the time band is a pre-determined period of time during which the specific complication is likely to occur following the procedure or treatment; designating the specific complication as related to the procedure or treatment only if the specific complication occurs during the time band for said complication; adjusting the designation of a specific complication as related to the procedure or treatment based on the number of patients at the outpatient facility having statistically significant risk factors and the risk odd ratios for such statistically significant risk factors; and deriving the predicted complication rate at each outpatient facility for each specific complication following the procedure or treatment by comparing the number of times the specific complication was related to the procedure or treatment to the total number of the procedures or treatments performed at the outpatient facility.

In some embodiments, the time band for the actual and/or predicted complication rate is a range of time determined by one of the following measures or a combination of one or more of the following measures performed by the computer system: (a) the average or mean time between the procedure or treatment and the occurrence of the particular complication for all of the outpatient facilities of the cohort; (ii) information regarding the timing of the specific complication for the procedure or treatment that the computer system scrapes from the internet, databases, and/or other electronic sources of information regarding the timing of the specific complication for the procedure or treatment; and/or (iii) input from one or more experts in the field of the procedure or treatment regarding the timing of the specific complication for the procedure or treatment. In some embodiments, the time band for the specific complication for measure (if) and/or (ii) is adjusted by input from one or more experts in the field of the procedure or treatment. In some embodiments, the range of the time band for measure (i) is based on a distribution of the time between the procedure or treatment and the occurrence of the particular complication for all of the outpatient facilities in the cohort. In other embodiments, the time band for the specific complication for measure (i) that is based on a distribution of the time between the procedure or treatment and the occurrence of the particular complication for all of the outpatient facilities in the cohort is adjusted by input from one or more experts in the field of the procedure or treatment.

In some embodiments, the computer system excludes from the determination of the actual and/or predicted complication rates the health data from patients with comorbidities and/or the health data from patients who underwent a deviation from the procedure or treatment that significantly affects the complication rate for the procedure or treatment.

In some embodiments, the computer system of the present invention may also determine the actual and/or predicted complication rate for the cohort of outpatient facilities as a whole for one or more specific complications relating to the procedure or treatment. Such actual complication rate for the cohort is determined by averaging the actual complication rates for each of the outpatient facilities belonging to the cohort, and the predicted complication rate for the cohort is determined by averaging the actual complication rates for each of the outpatient facilities belonging to the cohort.

In some embodiments, the computer system compares the actual complication rate for a specific complication related to the procedure or treatment for each outpatient facility to the actual complication rate for the specific complication for the cohort of outpatient facilities as a whole. Based on this comparison, the computer system may optionally assign a score to each outpatient facility for each of the specific complications related to the procedure or treatment. In another embodiment, the computer may list each outpatient facility in the cohort of outpatient facilities in order of the actual complication rate from lowest to highest actual complication rate or highest to lowest actual complication rate.

In some embodiments, the computer system compares the actual complication rate for a specific complication related to the procedure or treatment performed at each outpatient facility to the predicted complication rate for the specific complication related to the procedure or treatment performed at each outpatient facility. The computer system further may determine for each outpatient facility the ratio of the actual complication rate to the predicted complication rate.

In other embodiments, the computer system compares for the cohort of outpatient facilities as a whole, the actual complication rate for a specific complication related to the procedure or treatment performed at all of the outpatient facilities in the cohort to the predicted complication rate for the specific complication related to the procedure or treatment performed at all of the outpatient facilities in the cohort. The computer system further may determine the ratio of the actual complication rate to the predicted complication rate for the outpatient facility as a whole.

In some embodiments, the computer system compares for each selected complication related to the procedure or treatment, the ratio of the actual complication rate to the predicted complication rate for each of the outpatient facilities to the ratio of the actual complication rate to the predicted complication rate for the outpatient facilities as a whole. From this comparison, the computer system may optionally: (i) assign a score to each outpatient facility for each of the specific complications related to the procedure or treatment; (ii) list each outpatient facility in the cohort of outpatient facilities in order, such as from the lowest to the highest comparative rate or the highest to the lowest comparative complication rate.

Embodiments of the present application also relate to a method of identifying outpatient facilities in need of improvement where the computer system identifies the outpatient facilities as having a higher complication rate for a selected complication than the corresponding complication rate for the cohort of outpatient facilities as a whole.

Other embodiments include methods of identifying from the computer system those outpatient facilities performing the procedure or treatment with a lower complication rate for a selected complication than the corresponding complication rate for the cohort of outpatient facilities as a whole.

In general, the embodiments of the present application relate to a computer system that provides objective complication rate data from outpatient facilities. The foregoing disclosure provides illustration and description but is not intended to be exhaustive or to limit the aspects to the precise form disclosed. Modifications and variations may be made in light of the above disclosure or may be acquired from practice of the aspects.

In this application, a complication refers to any adverse event or negative medical condition that was not present before a procedure was performed. Additionally, outpatient facilities are defined as facilities where the patient stay is less than 24 hours, including acute care facilities such as hospitals as week as Ambulatory Surgical Care centers that perform “same-day” procedures. Moreover, the term “component” is intended to be broadly construed as hardware, firmware, or a combination of hardware and software. As used, a processor may be implemented in hardware, firmware, or a combination of hardware and software.

The foregoing has outlined rather broadly the features and technical advantages of the invention according to the disclosure. The conception and specific examples disclosed may be readily utilized as a basis for modifying or designing other structures to carry out the same purposes as the present disclosure. Such equivalent constructions do not depart from the scope of the appended claims. Characteristics of the concepts disclosed, both their organization and method of operation, together with associated advantages, will be better understood from the description when considered in connection with the accompanying figures. Each of the figures is provided for the purposes of illustration and description and not as a definition of the limits of the claims. Several aspects of the computer system and methods for the determination, comparison, and scoring of complication rates for outpatient facilities will now be presented with reference to various apparatuses and techniques. These will be described by various blocks, modules, components, circuits, steps, processes, algorithms, or the like, as well as illustrations and examples (collectively referred to as “elements”). These elements may be implemented using hardware, software, or combinations thereof. Whether such elements are implemented as hardware or software depends upon the particular application and design constraints imposed on the overall system.

It should be noted that while aspects may be described using various types of technologies, aspects of the present disclosure are not limited to a particular generation-based computer systems and can be applied in newer or older generation-based computer systems.

Additionally, a phrase referring to “at least one of” or “and/or” refers to any combination of those items, including single members. As an example, “at least one of: a, b, or c” is intended to cover a, b, c, a-b, a-c, b-c, and a-b-c, as well as any combination with multiples of the same element (for example, a-a, a-a-a, a-a-b, a-a-c, a-b-b, a-c-c, b-b, b-b-b, b-b-c, c-c, and c-c-c or any other ordering of a, b, and c).

Also, as used, the articles “a” and “an” are intended to include one or more items and may be used interchangeably with “one or more.” Furthermore, as used, the terms “set” and “group” are intended to include one or more items (for example, related items, unrelated items, a combination of related and unrelated items, or the like), and may be used interchangeably with “one or more.” Where only one item is intended, the phrase “only one” or similar language is used. Also, as used, the terms “has,” “have,” “having,” or the like are intended to be open-ended terms. Further, the phrase “based on” is intended to mean “based, at least in part, on” unless explicitly stated otherwise.

It will be apparent that systems or methods described may be implemented in different forms of hardware, firmware, or a combination of hardware and software. The actual specialized control hardware or software code used to implement these systems or methods is not limiting of the aspects. Thus, the operation and behavior of the system or methods are described without reference to specific software code—it being understood that software and hardware can be designed to implement the systems or methods based, at least in part, on the description.

Even though particular combinations of features are recited in the claims or disclosed in the specification, these combinations are not intended to limit the disclosure of various aspects. In fact, many of these features may be combined in ways not specifically recited in the claims or disclosed in the specification. Although each dependent claim listed below may directly depend on only one claim, the disclosure of various aspects includes each dependent claim in combination with every other claim in the claim set. No element, act, or instruction used should be construed as critical or essential unless explicitly described as such.

1. A method for rating a medical facility based on complication rates, comprising:
determining, for each procedure of a group of procedures, a temporal filter for complications associated with the procedure based on information obtained from a group of data sources, the group data sources including one or more data sources with encrypted patient data;
identifying a group of patients having performed a same procedure at the medical facility;
filtering, from the group of patients, complications associated with the group of patients based on the temporal filter to generate a list of filtered complications; and
generating a rating for the medical facility based on a quantity of complications in the list of filtered complications.

2. The method of claim 1, wherein the group of data sources include one or more of one or more internet sources, one or more scientific databases, one or more clinical databases, one or more journals, clinical trial data, one or more treatises, one or more training materials, one or more journal articles, one or more case studies, one or more clinical studies, studies patient health records, one or more medical billings claims data, and one or more expert statements.

3. The method of claim 1, wherein:
the temporal filter is determined by a first machine learning model that correlates patterns of complications to the procedure; and
the temporal filter is also associated with a patient demographic.

4. The method of claim 3, further comprising:
continuously monitoring one or more data sources of the group of data sources for an update;
reviewing, via a second machine learning model, the update to determine whether the update includes additional information regarding the procedure, the patient demographic, and/or the complications; and
updating, via the first machine learning model, the temporal filter based on the updating including additional information regarding the procedure, the patient demographic, and/or the complications.

5. The method of claim 1, wherein identifying the group of patients comprises:
receiving an outpatient record;
identifying one or more data gaps in the outpatient record;
identifying one or more data sources corresponding to the data gaps;
generating a list of data for filing the one or more data gaps;
generating a patient specific token;
transmitting the patient specific token and the list of data to the one or more data sources;
receiving data items corresponding to the list of data in according with transmitting the patient specific token and the list of data;
removing duplicate data from the data items; and
joining the data items and the outpatient record.

6. The method of claim 5, wherein the outpatient record includes one or more of patient demographic information, treatment or procedure information, diagnosis information, discharge status, and zero day complications.

7. The method of claim 1, wherein the medical facility is an outpatient facility.

8. An apparatus for rating a medical facility based on complication rates, comprising:
one or more processors; and
one or more memories coupled with the one or more processors and storing processor-executable code that, when executed by the one or more processors, is configured to cause the apparatus to:
determine, for each procedure of a group of procedures, a temporal filter for complications associated with the procedure based on information obtained from a group of data sources, the group data sources including one or more data sources with encrypted patient data;
identify a group of patients having performed a same procedure at the medical facility;
filter, from the group of patients, complications associated with the group of patients based on the temporal filter to generate a list of filtered complications; and
generate a rating for the medical facility based on a quantity of complications in the list of filtered complications.

9. The apparatus of claim 8, wherein the group of data sources include one or more of one or more internet sources, one or more scientific databases, one or more clinical databases, one or more journals, clinical trial data, one or more treatises, one or more training materials, one or more journal articles, one or more case studies, one or more clinical studies, studies patient health records, one or more medical billings claims data, and one or more expert statements.

10. The apparatus of claim 8, wherein:
the temporal filter is determined by a first machine learning model that correlates patterns of complications to the procedure; and
the temporal filter is also associated with a patient demographic.

11. The apparatus of claim 10, wherein execution of the processor-executable code further causes the apparatus tog:
continuously monitor one or more data sources of the group of data sources for an update;
review, via a second machine learning model, the update to determine whether the update includes additional information regarding the procedure, the patient demographic, and/or the complications; and
update, via the first machine learning model, the temporal filter based on the updating including additional information regarding the procedure, the patient demographic, and/or the complications.

12. The apparatus of claim 8, wherein execution of processor-executable code that causes the apparatus to identify the group of patients further causes the apparatus to:
receive an outpatient record;
identify one or more data gaps in the outpatient record;
identify one or more data sources corresponding to the data gaps;
generate a list of data for filing the one or more data gaps;
generate a patient specific token;
transmit the patient specific token and the list of data to the one or more data sources;
receive data items corresponding to the list of data in according with transmitting the patient specific token and the list of data;
remove duplicate data from the data items; and
join the data items and the outpatient record.

13. The apparatus of claim 12, wherein the outpatient record includes one or more of patient demographic information, treatment or procedure information, diagnosis information, discharge status, and zero day complications.

14. The apparatus of claim 8, wherein the medical facility is an outpatient facility.

15. A non-transitory computer-readable medium having program code recorded thereon for rating a medical facility based on complication rates, the program code executed by one or more processors and comprising:
program code to determine, for each procedure of a group of procedures, a temporal filter for complications associated with the procedure based on information obtained from a group of data sources, the group data sources including one or more data sources with encrypted patient data;
program code to identify a group of patients having performed a same procedure at the medical facility;
program code to filter, from the group of patients, complications associated with the group of patients based on the temporal filter to generate a list of filtered complications; and
program code to generate a rating for the medical facility based on a quantity of complications in the list of filtered complications.

16. The non-transitory computer-readable medium of claim 15, wherein the group of data sources include one or more of one or more internet sources, one or more scientific databases, one or more clinical databases, one or more journals, clinical trial data, one or more treatises, one or more training materials, one or more journal articles, one or more case studies, one or more clinical studies, studies patient health records, one or more medical billings claims data, and one or more expert statements.

17. The non-transitory computer-readable medium of claim 15, wherein:
the temporal filter is determined by a first machine learning model that correlates patterns of complications to the procedure; and
the temporal filter is also associated with a patient demographic.

18. The non-transitory computer-readable medium of claim 17, wherein execution of the processor-executable code further causes the apparatus tog:
continuously monitor one or more data sources of the group of data sources for an update;
review, via a second machine learning model, the update to determine whether the update includes additional information regarding the procedure, the patient demographic, and/or the complications; and
update, via the first machine learning model, the temporal filter based on the updating including additional information regarding the procedure, the patient demographic, and/or the complications.

19. The non-transitory computer-readable medium of claim 17, wherein the program code to identify the group of patients further includes:
program code to receive an outpatient record;
program code to identify one or more data gaps in the outpatient record;
program code to identify one or more data sources corresponding to the data gaps;
program code to generate a list of data for filing the one or more data gaps;
program code to generate a patient specific token;
program code to transmit the patient specific token and the list of data to the one or more data sources;
program code to receive data items corresponding to the list of data in according with transmitting the patient specific token and the list of data;
program code to remove duplicate data from the data items; and
program code to join the data items and the outpatient record.

20. The non-transitory computer-readable medium of claim 19, wherein the outpatient record includes one or more of patient demographic information, treatment or procedure information, diagnosis information, discharge status, and zero day complications.
